SEQUENCE LISTING (2 sequences)

SEQ ID NO: 1 - protein, 356 aa, Homo Sapiens
MGCPEVANCGSLGSSSRAGEVSSVSLNPGFGAMICRGVGVKDGRDLRVSPSRTWQRVLGTQRGEQGTLAAVGEDVAVDGQNWTFAFDFSFLSQQEDLAWAELRLQLSSPVDLPTEGSLAIEIFHQPKPDTEQASDSCLERFQMDLFTVTLSQVTFSLGSMVLEVTRPLSKWLKHPGALEKQMSRVAGECWPRPPTPPATNVLLMLYSNLSQEQRQLGGSTLLWEAESSWRAQEGQLSWEWGKRHRRHHLPDRSQLCRKVKFQVDFNLIGWGSWIIYPKQYNAYRCEGECPNPVGEEFHPTNHAYIQSLLKRYQPHRVPSTCCAPVKTKPLSMLYVDNGRVLLDHHKDMIVEECGCL

SEQ ID NO: 2 - DNA, 8294 bp, Homo Sapiens
ggtgacttcatcccacctccaaaatacatgcacatatgtctcaactttcacatacagtttcggggggttcacaggtttttaaaaaatccagtaaagaactcatgcacttgtgcttttccttgccactgtcttttcagtaaagagaacatccagccagccccctgcacaggcttcttcctccctcttcctgacagcagcctctgtgcttggccagactccactgagcccttcatttacagagtgggcagcccctcctcttcagttctggtgggcagagcaactttgcccctctctgtttctccttactggattagatggttatcatgtcttccaggagtttcccagccttccagagtgcaggcaaatccagtctccctccaggatgtcatcagaggcacccacattcttccacgatcatgtctttatggtgatctaggagcactctgccattatccacatacagcatgctcagcggcttggtcttcactggggcacaacaagtggaagggactcggtggggctggtaacgtttcagcagactctgtaaaggaaaggaagggtgtgtcaattcacatcctgatgggcaggtcagaatagtatttctgggtaaagagtaaaaaagttacggagttaaagtcagttcctgagtgcaaaaatagcagaaaattattttcataaaggaattagcttgccaatatatcatgaactctaatgtctaaactacctttagctctgtgcttgttttgtgaaaacagcctttaaacagattcaaaaaattacatgtatctgggtagagtagtagctatttttttttttaagtggtagctaatttttatgttctcctattattctagtctaaagggaataccattttttttttcttttttgagatggtcttgctctgttgcccaggctggagtgcagtggtgtgatcacagctcactgcatcctcaacctcctgggctcaggtgatcctcccacctcagcctcctgagtagctgggactacaggtacatgccaccatgcctggctaatttttgtattttttgtagaaatagggtttcggcatgttgcccaggctggtcttgacctcctgggctcaagcgatccacccacctcagcctcccaaaatgctggaattacatgagtgagccaccgtgcccggctgtgaataccaatttttaaaatattccaagaaattcctggtttagttctagaaatatgaagcagaatagttagtcatgtggaaattcttatgggatttgctgtcagccagagaaggacagagagagtcaataggaggaaaggaacaatcccttggcgttttggaaccagggcctgtccctgtggtctagtaacgtctttttagatcagagtgtcagagttggcagttgggactgtctgggtctttgtggtgctccaggtgggcgatgaaaatcagcaggcctgggtgaggctcaggacagatcctcggaagctctagtacccccagggactcccttgcatgacctcagcccatgtgctcatgctccccagagaccctgaatcccgcctgaggcttggcatggaggatatattgcaagtccaagaaattctcattccagacttcatggagtaaaatgaaaacattttggtcccatatgaccttggacagcagggcagggccacctataacatcagatccaccatattaggttatattttctgttctctccatcttcttctctttcctaactcaccagctctgttcttcctccaccacctcctagaccttaggctgactagtggcaccaagtggcagcctctggctttgtctactttttcttgtttttgagatggagtttcgctcctgttgcccaggctggagtgcaatggcatgatctcggctcactgcaatctctgcctcccgggttcaagtgattctcctgcctcagcctcccgagtagctgggattacaggcatgtgccacaacacccggctgatttttgtatttttagtagagacggggtgtcaccatgttggtcaggctggtctcaaactcctgacctcaggtgatccgcccacctcagcctcccaaagtgttgggatttcaggcatgtgccaccgcacctggctggccttgtctacttctgtagaatgggtagtcaattctcaaccaaaatactgtggctggaacagtgtgattccaaccgtggccccataatgcagccaaacatgcctgttacctaccaactgctttacatgccatgtgcatgtcatctcattaaccctacagcatccctctgaaatatggcattgccctcctcatcttacaggtggaaaaactgaagcttgagagaataagcaacctaaacagtatcacacagctaatgaggagtcaaatgcaggcctgcctgtccccaaagtccagattcttcctttcctagaatgtcagggctagggaagtgctgagaccctctggtctacccccaccccctaattttatgaaagacaaggataaagtcccagggaaacaatgtgttcaaggctacccagtgacgcatgaccccatacaggctctataaggatgttacactcggtcatctctacatgcctggtacctagcacaggatcctggtacattggaggtgcttgagtaactgtgaatacaggaacatagtcatttaatagcaaagctagagccctgtcccccaaggccagcttactgcctcccctccccctcacgcctggcatcccacctggatgtatgcatggttggtcggatgaaactcctccccaacaggattaggacactcgccctcacagcgataggcgttgtactgcttggggtagatgatccaggagccccatccgatcaggttgaagtccacctggaacttgaccttccgacacagttgacttctgtctggcaagtgatgtcgacggtgcctcttgccccactcccaggacagctgtccctcctgggcccgccaggagctctcggcttcccacagcaaggtggacccacccagctgcctctgctcctgcgagaggttggagtagagcataaggagcacattggtggcaggcggtgtggggggccgcggccagcactctccagctaccctggacatctgcttctccagggccccagggtgcttcagccacttggagagaggcctggtcacctccaaaaccatgctgcccaaggaaaaggtgacctgggacaaagtgacagtgaataggtccatctgaaaccgctctaagcagctgtctgaagcctgctctgtgtcgggctttggctggtggaaaatctcaatggcaagtgagccctcagtggggaggtccacagggctggacagctgcagccggagctcagcccatgccagatcctcttgttggctcaggaaggagaagtcaaaagcaaacgtccagttctgcccatccactgccacatctgggtaggagaaaaccaagaaggaaggagggtgagtgagggcctcccccagcctcagtcagtgtcacaaccaccatagctcttgctggaggccttaccttatatagaattaccttcgaattctcaccccaactctatgagttggtaagattttcgagtcccttttagagatgaggaaaactgaggctcagtgtggttaaaagatctgaccaaggtgtcattcttggaaggtggcagagccaagagtaaacctggatttgcccaactgccaagcccagactccgaattactgttcttccagtcccccttccaagataacttgcacagccagaggacagtcagtaaaaatgtgatgagttgcatctccccctcccctgtctcctcccatacatgaagtctgaccaacatggagaaaccctgtctctactaaaaatacaaaattagctgggcgtggtggtttgcgcctgtaatcccagccactcgagaatcatttgaacccgggaggcagaggttgcagtgagccaagacagtgccattgcactccagcctgcacaacaagagtgaaactctgtctcaaaaaaaaaaaaaaaaaagagtgacttgttttacagacacattgagctagatgtgaatatcatggaaaagctcagagctctcagggagccacaggcaatagaagccaaagactggtcaccctctacccaccccccagttcctaagagtttttggcaaccttacaaataggtttctaaaggctttctaggcatgtctacccacctacaggatgctcgcccattcctgtcccaggtcattcacacacctggggcacacagcaatcacatgcacccagaagcttccagaaggctgaaagtgggagttgcccctttacaatacaaggtggattatttcccaaagaaattcactttctctctgatcaaagatgctctatgagacaacagatgaataaactgttagatgcccagactggacctacatcagagatcatcacccagaagacacacacacccagccctgttccacacacccaagacaaccaaacccaagctggatgacaaggagtaggggagcacactccatttattctggtcctggattgattttccctgatgtctgggaatagcacagatttgtggattttcaggtgacttaatagtgatgccgggcgcggtggctcacgcctgtaatcccagcactttgggaggccaaggcgggtggatcacctgaggtcaggagttcgaaaccagcctgaccaacatggagaaaccccgtctctactaaaaatacaaaattagccgggtgtggtggctcacgcctaatcccagctactcaggaaggctgaggcaggagaattgcttgaacccaggaggtggaggttgtggtgagccgggatcgcgccactgaactctagcctgggcaacaagagcgaaactctgtctcaacaaaaacaacaacaaacatagtaatgaactttcttcaagctcagtttcctcttctttgcgatgggaataatgctgtaccaccctctctgattatcacaaggacccagagaaataaagaggtttgaaggtttattgctaaataaaagttggggttattcattagaaataagacagggcatttataattctttgtgaatagactctggaggtagtgatgatctctgatgtaggtccaggctggctgcataacagtttattcattgactgtctcgttgagcatctttggtcagagaggaagtgaatttctctgggaaatccagcttgtattgtaaaggggtcactcccaatctcagcctcatggcagcttctcccagcctatctccagcagctctttttcacttaatttccacttccctcctcattagcacctacaggaggcaataaccggcccctgactagcagctttccccagaagtgtggggaggaggctaacattactcagcactcactgctgccacctgtacctggaggattcgggcggtgagtttaaaaacactttatcttggccaggtgcggtgggtcactcctataatcccagcactttgggaggctgaggtgggtggatcacttgaggtcagaagttcgagaccagcctgatcaacatggtgaaaccctgtctctactaaaaaaaaaatacaaaattagccaggcatggtggtgcacacctgtaatcccagctacttgagaggcagaggcaggagaatcgcttgaacccaggaggtggaggttgcagtgagctgagattgcgccattgcactccagcctgggtgacgaaaagcaaaactctgtctcgaaaaaagaaaaactttatctcaactcttttagaagaaggcactcctgcttaatggttaagagcaggggcccccaagtctccccatgatggctcaaattctgtcacttgctgtttgactttggtcaggctactttccttctctgagcctcagtttccctctctgtaaaagggagatggtaaaagtagccacctcacaggggtgttaggattaactgagataattcatgtaatgtgccatgcccagcacaaccaaagttcaataaacattggccatcatcagctgcctcattattgcccaggtccgagagttagaaatgaaggtgtccggacaggccacaaggacctggacaggggtgcaaagtttcctatgaacaggatgggcagtgggaaaggctgggtagggaagaggctgctccttcctgctgaaagaagtctgtgacccttcctaccttctcccactgctgccagggtgccctggaacagggctgataggttctctggttcatcttagaataagggttctaatattgtgggtgtcactggtgcctgatgcaaggtaggaatgttatcagaaagaaacacacatccaaatacaacaaatgtgattttaaggtgggtatgaggaaggggttcatggacacccaaaaacccattcatatgctgaatgcagacttagccacacagatgcccctgatgtcctcctggcccacctccagttcttttttttgagatggagtctcgctctgtggccaggctcgagtgcagtggtgcgatctcagctcactgcagcctccacctcctgggttcaagtgattctcctgcctcaccctcccgagtagctgggactacaggtgcgcgccaccatgcccagctaatttttgtatttttagtagagacagggtttcatcatgttgggcaggacggtcttgatctcttgacctcatgatccgaccacctcggcctcccgaagtgctgggttacaggcgtaagccactgtgcccggccccacctccagttctgaagaagaaagtggctcacacccttgggttcccttccagtcaccgacaccgccgtccccagacagggctgtgaggcagggctgtaaggacctgacactgagcacccactctacattttcataaattataagggtcgttaccatcagtggcttcccgtgtgccaagtgtttcacaggcattctctaaatctcctcactgttcctctcatctcaccatttgcagatgagaaaactgaaggtccaaggtgaaggttaaacggcaacagatacagatgtaaacccaattggactccaacatccctttcatgcaactgcggtgcagcttattctaaataaacccaagatggaaggagtggggccaggacatcccttctcctccagtctccctccaggcgggctcttcccactgctcttagaaaatgaggccaattcacaacgtaactgtgccacatgccgctgtacactaaaaaacagtgtttaggccggtcacagtggctcacgcctgtaatcccaacaccttgggaggccgaggcaggcggaccacgagaccaggcgagaccagcctggccaatatggtgaaactctgtgtctactaaaaatattaaaaaatagctgggcgcagtggcgcgcgcctgtagtctcagttactcgggaggctgaggcaggagaattccttgaacctgggaggcggaggttgcagtgagccgaaatcgtgtcactgcactccagcctgggcaacagagggagactctgtttcaaagaaaaaacaaacaaacaaaaaaacagtgtttagctgaaagagagcagtctcctaatttacagaagggaaggcagataaaccaagacgcgccagggacaacagggaatcaggaagcaggatttgaggcctagaacccagtttccagatgcctgtccatcaccgcacacatctcgaactttccagaagggagtgaactggaaaagcgctccctcccttctttgccccctcccatccccggccgcttttcggttgaggggccgagcactccagcaaatttctcattgccccgcccccgagggtgtcccctggtctggctggtccccagatgacccttccggagagggtcaccggctgtctgaccccaggcgcgggagcgaagggcccccggcaccaggtcacaaggggcgggcaggcggcaggtcggatcagattagcgtttgtggattgcgccccggccccagggagggccgtctggcccccgcaggcggagggaggcgtgggcggccgcggctgcccaggcggcagaatgtggattgaggcgcggaaggggctccctctccgcagcacccacctgctcgccgcgctgggtgcccagaacgcgctgccaggtcctcgagggcgataccctcagatccctgccgtccttgacacccactcccctgcaaatcatggccccgaacccggggttaagcgagacacttgacacttccccggcgcggctggaggacccaaggctgccacagttcgcgacttcgggacagcccatc

(19) United States
(12) Patent Application Publication
(10) Pub. No.: US 2002/0123083 A1
(43) Pub. Date: Sep. 5, 2002

(54) Nucleic acid endocing growth factor protein

(75) Inventors: Ron T. Shigeta JR., Berkeley, CA (US); Michael A. Siani-Rose, San Francisco, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC.

(21) Appl. No.: 10/084,037

(22) Filed: Feb. 26, 2002

Related U.S. Application Data
(60) Provisional application No. 60/272,662, filed on Mar. 1, 2001.

(51) Int. Cl. G01N 33/574; A01K 67/00; G06F 19/00; G01N 33/48; C07H 21/04; C12P 21/02; C12N 5/06

(52) U.S. Cl. 435/7.23; 435/69.4; 435/325; 435/320.1; 800/8; 702/19; 536/23.5; 530/399

(57) ABSTRACT

The present invention provides a novel growth factor polynucleotide, growth factor polypeptide, gene delivery vehicles comprising and/or expressing the growth factor polynucleotide, antibodies and fragments capable of specifically binding to the growth factor polypeptide, receptors of the growth factor polypeptide, modulators of the growth factor activity, and modulators of growth factor expression. Also provided by the invention are host cells and transgenic organisms comprising the gene delivery vehicle of the present invention. Also provided by the invention are computer readable media containing the polynucleotide or polypeptide sequences of the present invention. Further provided are methods of using these compositions for diagnosis and treatment of growth factor associated diseases.

### CROSS-REFERENCE TO RELATED APPLICATION

This application claims priority from U.S. Ser. No. 60/272,662 filed Mar. 1, 2001, the disclosure of which is incorporated herein by reference.

### STATEMENT AS TO RIGHTS TO INVENTIONS MADE UNDER FEDERALLY SPONSORED RESEARCH AND DEVELOPMENT

Not applicable

### TECHNICAL FIELD

This invention is in the field of genetic analysis. Specifically, the invention relates to the discovery, identification and characterization of a nucleic acid that encodes a novel secreted growth factor. The compositions and methods embodied in the present invention are particularly useful for diagnosis, prognosis, drug screening, and/or treatment of disorders that are associated with dysfunction of the growth factors.

### BACKGROUND

Secreted cellular growth factors are molecules which bind to cell surface receptors to regulate a variety of cellular pathways. Growth factors have a demonstrated importance in a variety of cellular signaling pathways related to embryonic patterning events, cell cycle control, apoptosis, cellular differentiation, cell motility, and gene expression. In mammals, there are potentially hundreds of genes which encode growth factors of one type or another. Many of those growth factors are likely to be involved in various disease processes including, but not limited to, developmental and growth disorders, cardiovascular disorders, neurological and metabolic disorders, and various forms of cancer. Many therapeutic agents affecting growth factor functions and pathways have been successfully introduced onto the market. Clearly, growth factors are important diagnostic and/or therapeutic targets. There thus remains a considerable need for identification and characterization of novel growth factors.

### SUMMARY OF THE INVENTION

A principal aspect of the present invention relates to the discovery, isolation and characterization of novel growth factor polynucleotides, which exhibit sequence homology with previously characterized growth factors including:

HUMAN TRANSFORMING GROWTH FACTOR BETA (TGF-β) MURINE NODAL GENE PRODUCT

Novel growth factor polypeptides enable the discovery of other growth factors and growth factor receptors, and facilitate the study of cellular and gene expression pathways which are triggered or regulated by growth factors. Novel growth factors may be used to develop diagnostic and therapeutic tools related to growth factor related disorders.

In one embodiment, the present invention provides an isolated growth factor polynucleotide comprising a nucleic acid sequence depicted in FIG. 1B. In one aspect of this embodiment, the isolated polynucleotide comprising a nucleic acid sequence selected from the group consisting of: (a) a nucleic acid sequence of at least 90 nucleotides that is essentially identical to a linear sequence of comparable length contained in the sequence shown in FIG. 1B; (b) a nucleic acid sequence of at least 90 nucleotides encoding a polypeptide that is essentially identical to a linear sequence of at least 30 amino acids contained in the sequence shown in FIG. 1A; and (c) a complement of (a) or (b). In another aspect, the isolated polynucleotide encodes a polypeptide comprising an amino acid sequence that is essentially identical to a linear sequence of comparable length shown in FIG. 1A. In yet another aspect, the isolated polynucleotide encodes a polypeptide comprising an amino acid sequence essentially identical to the entire amino acid sequence shown in FIG. 1A. In still another aspect, the isolated polynucleotide encodes a polypeptide comprising the amino acid sequence shown in FIG. 1A. The polynucleotide of the present invention can code for the whole or domain(s) of the growth factor, or a mutant, fusion or a functionally equivalent growth factor polypeptide. In a related aspect of this embodiment, the invention encompasses a method of diagnosing a pathogenic condition or susceptibility to a pathogenic condition that is associated with a genetic alteration in a growth factor polypeptide encoded by the claimed polynucleotide. The method comprises the steps of: (a) providing a biological sample of a subject containing nucleic acid molecules and/or polypeptides; (b) determining a genetic alteration associated with the growth factor; and (c) correlating the alteration with a pathogenic condition or susceptibility to a pathogenic condition.

In another embodiment, the present invention includes a polynucleotide sequence that is useful as a probe for diagnostic or research purposes. Preferably, the probe is between 5 and 100 nucleotides in length and may comprise any of the contiguous nucleotides shown in FIG. 1A. Longer sequences may be used as probes depending on the type of assay used.

In a separate embodiment, the invention provides growth factor polypeptides encoded by the isolated polynucleotides. In another embodiment, the invention provides antibodies and antigen-binding fragments that are capable of specifically binding to the growth factor or fragments thereof. Also encompassed by the invention are gene delivery vehicles comprising the isolated growth factor polynucleotides, genetically engineered host cells and transgenic organisms carrying the gene delivery vehicles. Such a host cell or transgenic organism may express growth factor or lack growth factor expression (e.g. “knock-outs”). In a related aspect, the invention includes a recombinant method of producing a growth factor polypeptide that comprises culturing the genetically engineered host cell under conditions suitable for protein expression, and isolating the expressed polypeptide.

In another embodiment, this invention encompasses receptors and modulators including agonists and antagonists of growth factor. Modulators can be small molecules, large molecules, mutant growth factor receptors that compete with native natural growth factor receptor, and antibodies, as well as nucleotide sequences that can be used to inhibit growth factor gene expression (e.g., antisense and ribozyme molecules, and gene or regulatory sequence replacement constructs) or to enhance growth factor gene expression (e.g., expression constructs that place the growth factor gene under the control of a strong promoter system).

In yet another embodiment, the present invention provides a method for identifying a receptor or modulator that regulates growth factor expression or growth factor activity. Measurable growth factor activities include but are not limited to stimulation or inhibition of phospholipase C and adenyl cyclase, transient mobilization of Ca²⁺ from intracellular stores, ion flux, and change of intracellular pH condition.

In still another embodiment, the invention encompasses pharmaceutical compositions used for the diagnosis, prognosis or treatment of growth factor associated diseases.

Also provided by the invention are kits comprising the growth factor polynucleotides and/or polypeptides encoded thereby.

Further provided by the invention is a computer readable medium having recorded thereon the growth factor polynucleotide sequence and/or encoded gene product that are disclosed herein. The computer readable medium can be (a) magnetic storage medium; (b) optical storage medium; (c) electrical storage medium; or (d) hybrid storage medium of (a), (b) or (c).

### DETAILED DESCRIPTION

### A. General

The present invention relies on many patents, applications and other references for details known to those of the art. Therefore, when a patent, application, or other reference is cited or repeated below, it should be understood that it is incorporated by reference in its entirety for all purposes as well as for the proposition that is recited.

As used in the specification and claims, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, metazoans, or cells derived from any of the above.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example 1, 2, 3, 4, 5, and 6. This applies regardless of the breadth of the range.

### B. BRIEF DESCRIPTION OF THE DRAWINGS

FIG. 1 depicts nucleotide and protein sequence of the growth factor gene designated A.ctg13760-000001.3.0.

FIG. 1A depicts the amino acid sequence for the peptide encoded by polynucleotide A.ctg13760-000001.3.0.

FIG. 1B depicts the polynucleotide sequence of A.ctg13760-000001.3.0.

### C. DEFINITIONS

The terms “polynucleotide,” “nucleotide,” “nucleic acid,” and “oligonucleotide” are used interchangeably. They refer to any polymeric form of pyrimidine and purine bases, preferably cytosine, thymine, and uracil, and adenine and guanine, respectively. See Albert L. Lehninger, PRINCIPLES OF BIOCHEMISTRY, at 793-800 (1982). Such polymeric forms may be of any length, either deoxyribonucleotides or ribonucleotides, peptide nucleic acid components, and any chemical variants thereof, such as methylated, hydroxymethylated, or glycosylated forms of these bases and the like. Modifications to the nucleotide structure may be imparted before or after assembly of the polymer. Polynucleotides may have any three-dimensional structure and may perform any function, known or unknown. Polynucleotides may be heterogeneous or homogeneous in composition and may be isolated from naturally-occurring sources or may be artificially or synthetically produced. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. The sequence of nucleotides may be interrupted by non-nucleotide components. A polynucleotide may be further modified after polymerization, such as by conjugation with a labeling component.

A “nucleotide probe” or “probe” refers to a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, polynucleotides, nucleic acids, oligosaccharides, proteins and monoclonal antibodies.

A “primer” is a short oligonucleotide, generally with a free 3′-OH group, capable of acting as a point of initiation for template-directed DNA synthesis under suitable conditions, e.g. buffer and temperature, in the presence of four different nucleoside triphosphates and an agent for polymerization, DNA or RNA polymerase or reverse transcriptase. The length of the primer, in any given case, depends on, for example, the intended use of the primer, and generally ranges from 15 to 30 nucleotides, and all lengths in between. Short primer molecules generally require cooler temperatures to form sufficiently stable hybrid complexes with the template. A primer need not reflect the exact sequence of the template, but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

“Operably linked” or “operatively linked” refers to a juxtaposition wherein the components so described are in a relationship permitting them to function in their intended manner. For instance, a promoter sequence is operably linked to a coding sequence if the promoter sequence promotes transcription of the coding sequence. Generally, “operably linked” means that DNA sequences being linked are contiguous and, where necessary to join two peptide coding regions, in reading frame with one another.

A “gene” refers to a polynucleotide containing at least one open reading frame and capable of encoding a functional product. Functional products include, but are not limited to, mRNA, tRNA, rRNA, or polypeptides.

The term “isolated,” as used herein, means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragment thereof, does not require “isolation” to distinguish it from its naturally occurring counterpart. In addition, a “concentrated,” “separated,” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or, for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature. Methods for producing, isolating and purifying polypeptides are known in the art. See Murray P. Deutscher, et. al. ed., GUIDE TO PROTEIN PURIFICATION: METHODS IN ENZYMOLOGY, v.182, (Academic Press, 1997) and Paul Lloyd-Williams, CHEMICAL APPROACHES TO THE SYNTHESIS OF PEPTIDES AND PROTEINS, (1997). See also U.S. Pat. No. 5,419,899 (Koths, et. al.)

A “disease-associated” gene or polynucleotide refers to any gene or polynucleotide which is yielding transcription or translation products at an abnormal level or in an abnormal form in cells derived from disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Different polynucleotides are said to “correspond” to each other if one is ultimately derived from another. For example, a sense strand corresponds to the anti-sense strand of the same double-stranded sequence. mRNA (also known as gene transcript) corresponds to the gene from which it is transcribed. cDNA corresponds to the RNA from which it has been produced, such as by a reverse transcription reaction, or by chemical synthesis of a DNA based upon knowledge of the RNA sequence. A cDNA also corresponds to the gene that encodes the RNA. A polynucleotide may be said to correspond to a target polynucleotide even when it contains a contiguous portion of the sequence that shares substantial sequence homology with the target sequence when optimally aligned.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectively referred to as the “gene product.” If the polynucleotide is derived from genomic DNA, expression may include splicing of the mRNA in an eukaryotic cell.

“Differentially expressed,” as applied to nucleotide sequence or polypeptide sequence in a subject, refers to over-expression or under-expression of that sequence when compared to that detected in a control. Underexpression also encompasses absence of expression of a particular sequence as evidenced by the absence of detectable expression in a test subject when compared to a control.

“Differential expression” or “differential representation” refers to alterations in the abundance or the expression pattern of a gene product. An alteration in “expression pattern” may be indicated by a change in tissue distribution, or a change in hybridization pattern reviewed on a polynucleotide microarrays.

In the context of polynucleotides, a “linear sequence” or a “sequence” is an order of nucleotides in a polynucleotide in a 5′ to 3′ direction in which residues that neighbor each other in the sequence are contiguous in the primary structure of the polynucleotide. A “partial sequence” is a linear sequence of part of a polynucleotide which is known to comprise additional residues in one or both directions.

A linear sequence of nucleotides is “identical” to another linear sequence, if the order of nucleotides in each sequence is the same, and occurs without substitution, deletion, or material substitution. It is understood that purine and pyrimidine nitrogenous bases with similar structures can be flnctionally equivalent in terms of Watson-Crick base-pairing; and the inter-substitution of like nitrogenous bases, particularly uracil and thymine, or the modification of nitrogenous bases, such as by methylation, does not constitute a material substitution. A RNA and a DNA polynucleotide have identical sequences when the sequence for the RNA reflects the order of nitrogenous bases in the polyribonucleotides, the sequence for the DNA reflects the order of nitrogenous bases in the polydeoxyribonucleotides, and the two sequences satisfy the other requirements of this definition. Where one or both of the polynucleotides being compared is double-stranded, the sequences are identical if one strand of the first polynucleotide is identical with one strand of the second polynucleotide. It is understood that nucleic acid analogues are included in this definition.

The term “hybridize” as applied to a polynucleotide refers to the ability of the polynucleotide to form a complex that is stabilized via non-covalent bonding, usually hydrogen bonding, between bases of the nucleotide residues. The hydrogen bonding may occur by Watson-Crick base pairing, Hoogstein binding, or in any other sequence-specific manner. The resulting complex is referred to as a “hybrid” which may comprise two strands forming a duplex structure, three or more strands forming a multi-stranded complex, a single self-hybridizing strand, or any combination of these. The hybridization reaction may constitute a step in a more extensive process, such as the initiation of a PCR reaction, or the enzymatic cleavage of a polynucleotide by a ribozyme. The proportion of the population of polynucleotides that form stable hybrids is referred to as the “degree of hybridization.”

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

Melting temperature of a primer refers to the temperature at which 50% of the primer-template duplexes are dissociated. Melting temperature is a function of ionic strength, base composition, and the length of the primer. It can be calculated using either of the following equations:

Tₘ(° C.)=81.5+16.6×log[Na]+0.41×(% GC)−600/N

where [Na] is the concentration of sodium ions, and the % GC is in number percent, where N is chain length, or

Tₘ(° C.)=2×(A+T)+4×(C+G)

where A, T, G and C represent the number of adenosine, thymidine, guanosine and cytosine residues in the primer.

“In situ hybridization” is a well-established technique that allows specific polynucleotide sequences to be detected in morphologically preserved chromosomes, cells or tissue sections. In combination with immunocytochemistry, in situ hybridization can relate microscopic topological information to gene activity at the DNA, mRNA and protein level.

“Signal transduction” is a process during which stimulatory or inhibitory signals are transmitted into and within a cell to elicit an intracellular response. A “modulator of a signal transduction pathway” refers to a compound which modulates the activity of one or more cellular proteins mapped to the same specific signal transduction pathway. A modulator may augment or suppress the activity of a signaling molecule.

The terms “polypeptide,” “peptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, through disulfide bond formation, glycosylation, lipidation, acetylation, phosphorylation, or any other manipulation, such as conjugation with a labeling component. As used herein, the term “amino acid” refers to either naturally-occurring and/or artificial or synthetic amino acids, including glycine, D and/or L optical isomers, amino acid analogs and peptidomimetics. Standard abbreviations for amino acids are used (e.g. P for proline). These abbreviations are included in Lubert Stryer, BIOCHEMISTRY, (4^{th} ed. 1995), which is incorporated herein by reference for all purposes.

A “ligand” refers to a molecule capable of being recognized and bound by an ligand binding entity, such as a receptor. The molecule may be naturally-occurring and/or artificial or synthetic. The term ligand does not imply any particular molecular size or other structural or compositional feature other than the ability to bind or otherwise interact with the ligand binding entity. Examples of ligands include, but are not limited to, agonists and antagonists for cell-membrane receptors, toxins and venoms, viral epitopes, hormones (e.g. steroids, opiates, etc.), hormone receptors, peptides, enzymes, enzyme substrates, substrate analogs, transition state analogs, cofactors, drugs, proteins, and antibodies.

“Receptors” or “antigens” are molecules with an affinity for a given ligand. An “antigen” as used herein means a substance recognized and bound specifically by an antibody, a fragment thereof or by a T cell antigen receptor. Antigens can include peptides, proteins, glycoproteins, polysaccharides and lipids; portions thereof and combinations thereof. The antigens can be those found in nature or can be synthetic. They may be present on the surface or located within a cell. “Cell surface receptors” or “surface antigens” are molecules anchored on the cell plasma membrane. They constitute a large family of proteins, glycoproteins, polysaccharides and lipids, which serve not only as structural constituents of the plasma membrane, but also as regulatory elements governing a variety of biological functions. Receptors may be attached, covalently or noncovalently, to a binding member, either directly or via a specific binding substance. Examples of receptors which can be employed or investigated by this invention include, but are not restricted to, antibodies, cell membrane receptors, monoclonal antibodies and antisera reactive with specific antigenic determinants (such as on viruses, cells, or other materials), drugs, polynucleotides, nucleic acids, peptides, cofactors, lectins, sugars, polysaccharides, cells, cellular membranes, and organelles. Receptors are sometimes referred to in the art as ligand binding entities. As the term receptor is used herein, no difference in meaning is intended.

As used in this invention, the term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

As used herein, “membrane proteins” include peripheral and integral membrane polypeptides that are bound to any cellular membranes including plasma membranes and membranes of intracellular organelles.

A “database” is a collection of data which has some common or distinct characteristics.

A “genetically engineered host cell” includes an individual cell or cell culture which can be or has been a recipient for vector(s) or for incorporation of nucleic acid molecules and/or proteins. Host cells include progeny of a single host cell, and the progeny may not necessarily be completely identical (in morphology or in genome of total DNA complement) to the original parent cell due to natural, accidental, or deliberate mutation. A host cell includes cells transfected in vivo with a polynucleotide(s) of this invention.

“Luminescence” is the term commonly used to refer to the emission of light from a substance for any reason other than a rise in its temperature. In general, atoms or molecules emit photons of electromagnetic energy (e.g., light) when then move from an “excited state” to a lower energy state (usually the ground state); this process is often referred to as “radioactive decay.” There are many causes of excitation. If exciting cause is a photon, the luminescence process is referred to as “photoluminescence.” If the exciting cause is an electron, the luminescence process is referred to as “electroluminescence.” More specifically, electroluminescence results from the direct injection and removal of electrons to form an electron-hole pair, and subsequent recombination of the electron-hole pair to emit a photon. Luminescence resulting from a chemical reaction is usually referred to as “chemiluminescence.” Luminescence produced by a living organism is usually referred to as “bioluminescence.” If photoluminescence is the result of a spin-allowed transition (e.g., a singlet-singlet transition, triplet-triplet transition), the photoluminescence process is usually referred to as “fluorescence.” Typically, fluorescence emissions do not persist after the exciting cause is removed as a result of short-lived excited states which may rapidly relax through such spin-allowed transitions. If photoluminescence is the result of a spin-forbidden transition (e.g., a triplet-singlet transition), the photoluminescence process is usually referred to as “phosphorescence.” Typically, phosphorescence emissions persist long after the exciting cause is removed as a result of long-lived excited states which may relax only through such spin-forbidden transitions. A “luminescent label” may have any one of the above-described properties.

“Subject,” “individual” and “patient,” used interchangeably herein, refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Eric W. Martin, ed., REMINGTON'S PHARM. Sci., (15^{th} ed. 1975).

“Gene delivery vehicle,” “recombinant cloning vector,” and “recombinant expression vector” are used interchangeably and refer to a DNA or RNA molecule that encodes a functional product which may include, but is not limited to, mRNA, tRNA, rRNA, or a polypeptide, which has been inserted into any molecule that can carry such inserted polynucleotides into a host cell. For the purpose of the present invention, a cloning vector typically serves primarily as an intermediate in the construction of an expression vector; the latter vector is used to transform or transfect a host cell (or is introduced into a cell-free transcription and translation system) so that the encoded functional product is produced. Examples of gene delivery vehicles include, but are not limited to, retroviruses, bacteriophage, cosmids, plasmids, fungal vectors, and other recombination vehicles typically used in the art which have been described for expression in a variety of eukaryotic and prokaryotic hosts and may be used for gene therapy as well as for simple RNA or protein expression.

A “viral vector” is defined as a recombinantly produced virus or viral particle that comprises a polynucleotide to be delivered into a host cell, either in vivo, ex vivo or in vitro. Examples of viral vectors include retrovirus vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a therapeutic gene. Adenoviruses (Ads) are a relatively well characterized, homogenous group of viruses, including over 50 serotypes. (see e.g., WO 95/27071) Ads are easy to grow and do not require integration into the host cell genome. Recombinant Ad-derived vectors, particularly those that reduce the potential for recombination and generation of wild-type virus, have also been constructed. (see WO 95/00655; WO 95/11984). Wild-type AAV has high infectivity and specificity integrating into the host cells genome. (Hermonat, P. L. and Muzyczka, N. (1984) PNAS USA 81:6466-6470; Lebkowski, J. S. et al. (1988) Mol. Cell. Biol. 8:3988-3996).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add, or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

### D. MODES(S) FOR CARRYING OUT THE INVENTION

### General Techniques

The practice of the present invention will employ, unless otherwise indicated, conventional techniques of immunology, biochemistry, chemistry, molecular biology, microbiology, cell biology, genomics and recombinant DNA, which are within the skill of the art. See, e.g., Matthews, R. E. F., FUNDAMENTALS OF PLANT VIROLOGY (3^{rd} ed. 1991); Sambrook, J. et al., MOLECULAR CLONING: A LABORATORY MANUAL, (3^{rd} ed. 2001); Ausubel, F. M. et al., CURRENT PROTOCOLS IN MOLECULAR BIOLOGY (1987); METHODS IN ENZYMOLOGY (Academic Press, Inc.): MacPherson, M. J. et al., eds., PCR 2: A PRACTICAL APPROACH (1995); Deutscher, M. P. et al., eds. GUIDE TO PROTEIN PURIFICATION (1997), Harlow, E. and Lane, D, eds. ANTIBODIES, A LABORATORY MANUAL (1988); and Freshney, R. I., ed. CULTURE OF ANIMAL CELLS: A MANUAL OF BASIC TECHNIQUE (4^{th} ed. 2000).

### POLYNUCLEOTIDES OF THE PRESENT INVENTION

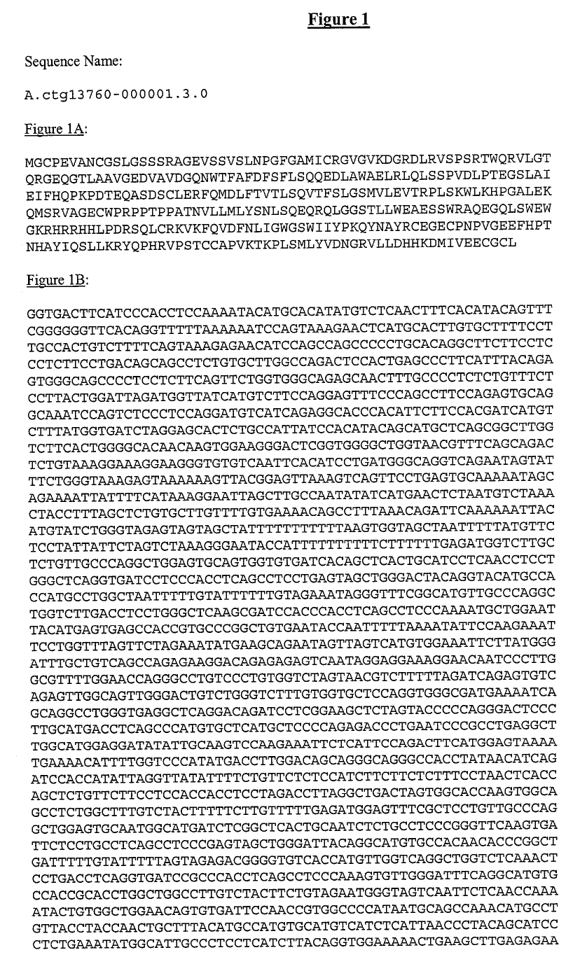

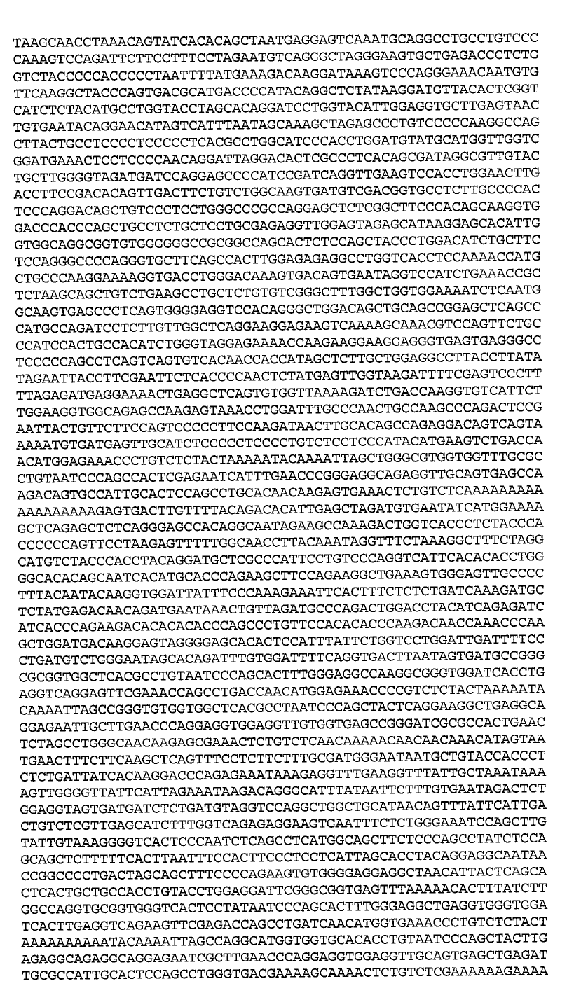

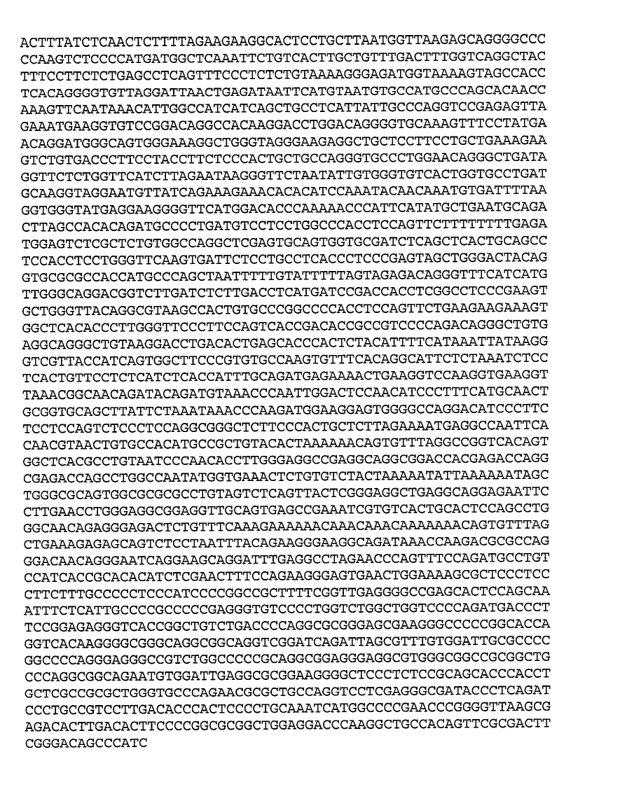

A central aspect of the present invention is the discovery of a novel growth factor gene comprising a nucleic acid sequence depicted in FIG. 1. In one aspect, novel growth factor polynucleotides can be identified by first building Hidden Markov Models (hMMs) for sub-classes of growth factors using SAM 3.0 (available from University of California, Santa Cruz), target-99 script as described in Hughey et al. (SAM: Sequence Alignment and Modeling Software System Technical Report UCSC-CRL-95-7, University of California, Santa Cruz, Computer Engineering). General methodology for building Hidden Markov Models are described Krogh, A. et al. (1994) J Mol Biol 235(5):1501-31; Brown, et. al. (1 993) Proc Int Conf Intell Syst Mol Biol 1:47-55; and Baldi, P. and Chauvin, Y. (1994) J Comput Biol 1(4):311-336. The next step is to screen candidate gene sequences using SAM3.0 hMM score algorithm, or the like, against the hMMs built for a specific class of growth factors. An alignment using SAM 3.0 align2model was performed using the best hits against this hMMS. In one aspect, the high-scoring protein sequences which match the hMM set are sorted so that the top 25, preferably top 10, more preferably top 5 entries have (a) no corresponding sequences in the non-redundant set (NCBI); and (b) no significant homology with hMMS for other families of proteins. Refinement of gene using GeneWise (Birney, E. et al. Genome Res (4):547-548(2000)) to map a known protein onto the portion of the genomic DNA. Each of the novel genes that match the family 3946 hMM best were searched against the GenBank non-redundant database (All non-redundant GenBank CDS translations+PDB+SwissProt+PIR+PRF) using BLASTed (Altschul) program to find close homologs. The nearest human homolog (or non-human) when there is no human entry) is then used as a probe for GeneWise to match against the portion of the contig from which the original gene was derived. For example, if A.ctg15361-000002.13.0 is derived from contig 15361 of the human genome, a portion 50,000 base pairs longer in each direction from the predicted gene is extracted from the contig. Genewise then attempts to match the protein sequence against the genomic sequence best, defining the gene structure (introns and exons) based on the homolog and its knowledge of gene structure in general.

In a separate embodiment, the present invention provides an isolated polynucleotide comprising a nucleic acid sequence having at least about 90 nucleotides that is essentially identical to a linear sequence of comparable length contained in the sequence shown in 1B. Preferably, the isolated polynucleotide contains at least about 90 nucleotide bases, more preferably at least about 150 nucleotides, more preferably at least about 450 nucleotides, and even more preferably at least about 1200 nucleotides. When the polynucleotide sequence is used as a probe, then it can also be shorter in length. For example, the sequence can be any contiguous nucleotides along the sequence shown in FIG. 1B, its complement, or a variation of a few nucleotides. The length can be from 5, 13, 15, or 20 nucleotides to 25, 30, 50, 75, 100 or more nucleotides in length. In some embodiments very long sequences can be physically attached to a substrate that may be 500 to 5,000 or even 50,000 nucleotides long.

In another embodiment, the isolated polynucleotide comprises a nucleic acid sequence of at least 90 nucleotides that encodes a polypeptide essentially identical to a linear sequence of at least 30 amino acids depicted in FIG. 1A. Preferred linear peptide sequence is at least about 50 amino acids in length, more preferably at least 150 amino acids in length, and more preferably at least 350 amino acids. In yet another embodiment, the isolated polynucleotide may be any polynucleotide which encodes the polypeptide of FIG. 1A. In yet another embodiment, the isolated polynucleotide is a complement of any of the above mentioned growth factor polynucleotides.

These gene sequences can be identified, in whole or in part, by specifically hybridizing under moderate or stringent conditions to the exemplary polynucleotides shown in FIG. 1B. Alternatively, the invention sequences can be identified by their homology to published or known open reading frames, or pieces of genomic sequences using computer-assisted methods known in the art or those described herein.

Thus, in one aspect, a linear sequence of nucleotides is “essentially identical” to another linear sequence, if both sequences are capable of hybridizing to form a duplex with the same complementary polynucleotide. The term “hybridize” as applied to a polynucleotide refers to the ability of the polynucleotide to form a complex that is stabilized via hydrogen bonding between the bases of the nucleotide residues in a hybridization reaction. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. The hybridization reaction may constitute a step in a more extensive process, such as the initiation of a PCR reaction, or the enzymatic cleavage of a polynucleotide by a ribozyme.

Hybridization can be performed under conditions of different “stringency.” Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. In general, a low stringency hybridization reaction is carried out at about 40° C. in about 10× SSC (or a solution of equivalent ionic strength/temperature. A moderate stringency hybridization is typically performed at about 50° C. in about 6× SSC, and a high stringency hybridization reaction is generally performed at about 60° C. in about 1× SSC.

Sequences that hybridize under conditions of greater stringency are more preferred. As is apparent to one skilled in the art, hybridization reactions can accommodate insertions, deletions, and substitutions in the nucleotide sequence. Thus, linear sequences of nucleotides can be essentially identical even if some of the nucleotide residues do not precisely correspond or align. In general, essentially identical sequences of about 60 nucleotides in length will hybridize at about 50° C. in 10× SSC; preferably, they will hybridize at about 60° C. in 6× SSC; more preferably, they will hybridize at about 65° C. in 6× SSC; even more preferably, they will hybridize at about 70° C. in 6× SSC, or at about 40° C. in 0.5× SSC, or at about 30° C. in 6× SSC containing 50% formamide; still more preferably, they will hybridize at 40° C. or higher in 2× SSC or lower in the presence of 50% or more formamide. It is understood that the rigor of the test is partly a function of the length of the polynucleotide; hence shorter polynucleotides with the same homology should be tested under lower stringency and longer polynucleotides should be tested under higher stringency, adjusting the conditions accordingly. The relationship between hybridization stringency, degree of sequence identity, and polynucleotide length is known in the art and can be calculated by standard formulae.

Sequence homology or identity can also be determined with the aid of computer methods. A variety of sequence analysis software programs are available in the art. Non-limiting examples of these programs are Bestfit program (Wisconsin Sequence Analysis Package, Genetics Computer Group, Madison Wis.), Fasta (Wisconsin Sequence Analysis Package, Genetics Computer Group, Madison Wis.), Blast (http://www.ncbi.nlm.nih.gov/BLAST/), DNA Star, MegAlign, GeneJocky, CLUSTAL W (Nucleic Acids Research 22: 4673-4680 (1994)) and SAM (Hughey et al. Technical Report UCSC-CRL-95-7, University of California, Santa Cruz, Computer Engineering(1995)). Sequence similarity is typically discerned by comparing a query sequence (polynucleotide or polypeptide sequence) to a reference sequence or a plurality of reference sequences contained in a database. Any public or proprietary sequence databases that contain DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS. Common parameters for determining the extent of homology set forth by one or more of the aforementioned alignment programs include p value and percent sequence identity. P value is the probability that the alignment is produced by chance. For a single alignment, the p value can be calculated according to Karlin et al. (1990) Prco. Natl. Acad. Sci 87: 2264-2268. For multiple alignments, the p value can be calculated using a heuristic approach such as the one programmed in Blast. Percent sequence identity is defined by the ratio of the number of nucleotide or amino acid matches between the query sequence and the reference when the two are optimally aligned.

Polynucleotides that correspond or align more closely to the exemplary sequences disclosed herein are comparably more preferred. A query polynucleotide of at least 90 nucleotides is considered to be essentially identical to a reference polynucleotide (e.g. sequences shown in 1B.), when the query polynucleotide exhibits at least about 80% sequence identity, more preferably at least about 90% identity, even more preferably at least about 95% identity using any of the above-mentioned alignment programs with the default settings. Likewise, a query polypeptide is essentially identical to a reference polypeptide of at least 30 amino acids, when the query polypeptide shares at least 80% sequence identity, more preferably at least about 90% identity, even more preferably at least about 95% identity that can be discerned by the aforementioned programs using their respective default settings. When using Bestfit or any other sequence alignment program to determine whether a particular sequence is, for example, 80% identical to a reference sequence of the present invention, the percentage of identity is preferably calculated over a linear sequence of comparable length that is contained in the reference sequence. Typically, the upper limit of gaps in homology is set at 20% of the total number of amino acid residues or nucleotide residues in the respective reference sequence. The altered residues may occur at the amino or carboxyl terminal positions of the reference sequence or anywhere between those terminal positions, interspersed either individually among residues in the reference sequence or in one or more contiguous groups within the reference sequence. Allowable sequence alterations include but are not limited to deletion, insertion, translocation and substitution of individual residues.

Polynucleotides of the present invention also include polynucleotides resulting from the alteration of codons to enhance expression of the growth factor. Examples of such alterations include, but are not limited to, substitutions of rare codons for common codons in a given host. For example, substitution of a codon commonly used in humans but rarely used in bacteria for a codon which is more common in bacteria to enhance bacterial expression is envisioned by this invention. Such codon substitutions to encode an identical polypeptide are well known in the art.

Essentially identical nucleic acid sequences can also be characterized as possessing essentially the same functionality of the exemplary nucleic acids. Functionality may be established by different criteria, which includes the ability to, (a) hybridize with a target polynucleotide; (b) effectively amplify a target sequence to yield a substantially homogenous multiplicity of products; (c) extend the 3′ end sequence complementary to a target sequence in a nucleotide sequencing reaction; and (d) function in substantially the same manner to produce essentially the same protein product as the nucleic acid exemplified herein, by virtue of the degeneracy of the genetic codes, or that have conservative amino acid substitutions. Suitable amino acid substitutions may be made on the basis of similarity in polarity, charge, solubility, hydrophobicity, hydrophilicity, and/or the amphipathic nature of the residues as long as the functional characteristics of the growth factor polynucleotide is retained. For instance, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine and; phenylalanine and tyrosine

The polynucleotides of the present invention can also comprise heterologous sequences that are not naturally found linked to the sequences shown in FIG. 1B. The choice of heterologous sequences is largely dependent on the intended purpose. Where desired, the heterologous sequence may encode a polypeptide that facilitates detection of the expression and purification of the gene product. Examples of such sequences are known in the art and include those encoding reporter proteins such as β-galactosidase, β-lactamase, chloramphenicol acetyltransferase (CAT), luciferase, green fluorescent protein (GFP) and their derivatives. Other heterologous sequences that facilitate purification include epitopes such as Myc, HA (derived from influenza virus hemagglutinin), His-6, FLAG, glutathione S-transferase (GST), maltose-binding protein (MBP), or the Fc portion of immunoglobulin. The heterologous sequences may also code for polypeptides that direct the intracellular localization of the expressed gene product. Examples of this class of heterologous sequences include but are not limited to those codes for a leader sequence that effects secretion of the gene product; membrane localization signal sequence that anchors a protein to the intracellular membranous structures, such as plasma membrane, nucleus, Golgi apparatus, endoplasmic reticulum, endosome, lysosome, and mitochondria. One skilled in the art can readily fashion a vast diversity of heterologous sequences based on the wealth of genetic data available in the art.

The polynucleotides embodied in the invention also include nucleotide sequences that encode full-length growth factor, mutant growth factor, peptide fragments of the full-length growth factor, truncated growth factor, and growth factor fusion proteins. These include, but are not limited to nucleotide sequences encoding mutant growth factor isolated by the methods disclosed herein; polypeptides or truncated peptides. Nucleotides encoding fusion proteins may include, but are not limited to, full length growth factor, truncated growth factor, or peptide fragments of growth factor fused to an unrelated protein or peptide, such as, for example, an Ig Fc domain which increases the stability and half life of the resulting fusion protein (e.g., growth factor-Ig) in the bloodstream; or an enzyme, fluorescent protein, luminescent protein which can be used as a marker. See U.S. Pat. Nos. 6,114,146 (Herlitschka, et al.); 5,602,034 (Tekamp-Olson); 5,238,820 (Kaufman); 5,013,653 (Huston, et al.).

Larger gene fragments containing or corresponding to the growth factor polynucleotides of the invention can readily be isolated, without undue experimentation using a variety of recombinant DNA techniques. These large fragments can be full-length growth factor cDNAs, mutant growth factor polynucleotides, splice variants of the exemplary growth factor, or growth factor homologs expressed in other species. The identification of homologs of growth factor in related species can be useful for developing animal model systems more closely related to humans for purposes of drug discovery. For example, expression libraries of cDNAs synthesized from mRNA derived from the organism of interest can be screened using labeled natural growth factor receptors derived from that species, e.g., a synthetic or natural growth factor receptor fusion protein. Alternatively, such cDNA libraries, or genomic DNA libraries derived from the organism of interest can be screened by hybridization using the polynucleotides described herein as hybridization or amplification probes. Furthermore, genes at other genetic loci within the genome that encode proteins which have extensive homology to one or more domains of the growth factor gene product can also be identified via similar techniques. For cDNA libraries, such screening techniques can identify clones derived from alternatively spliced transcripts in the same or different species.

Expression libraries may also be employed to screen for homologs, mutants or splice variants. An expression library can typically be constructed utilizing cDNA synthesized from, for example, RNA isolated from a tissue known, or suspected, to express a mutant growth factor allele in an individual suspected of or known to carry such a mutant allele. In this manner, gene products made by the putatively mutant tissue may be expressed and screened using standard antibody screening techniques in conjunction with antibodies raised against the normal growth factor gene product, as described, below. For screening techniques, see, for example, Harlow, E. and Lane, D, eds. ANTIBODIES, A LABORATORY MANUAL (1988). Additionally, screening can be accomplished by screening with labeled natural or synthetic growth factor receptor or receptor fusion proteins. In cases where a growth factor mutation results in an expressed gene product with altered finction (e.g., as a result of a missense or a frameshift mutation), a polyclonal set of antibodies to the growth factor are likely to cross-react with the mutant growth factor gene product. Library clones detected via their reaction with such labeled antibodies can be purified and subjected to sequence analysis according to methods well known to those of skill in the art.

The cDNA or genomic library screens can be carried out on arrays immobilized with candidate sequences. Suitable arrays include conventional filters, DNA microarrays such as DNA arrays manufactured by Affymetrix which are sold under the name GeneChip®. Probe arrays that are made by depositing larger sequences by “spotting” can also be employed. See U.S. Pat. No. 6,040,193. The labeled probe may contain at least about 15-30 base pairs of the growth factor nucleotide sequence, as shown in FIG. 1B. The hybridization washing conditions used should be of a lower stringency when the cDNA library is derived from an organism different from the type of organism from which the labeled sequence was derived.

Low stringency conditions are well known to those of skill in the art, and will vary predictably depending on the specific organisms from which the library and the labeled sequences are derived. For guidance regarding such conditions see, for example, Sambrook, J. et al., MOLECULAR CLONING: A LABORATORY MANUAL, (3^{rd} ed. 2001); Ausubel, F. M. et al., CURRENT PROTOCOLS IN MOLECULAR BIOLOGY (1987).

Alternatively, the labeled growth factor nucleotide probe may be used to screen a genomic library derived from the organism of interest, using appropriately stringent conditions. The identification and characterization of human genomic clones is helpful for designing diagnostic tests and clinical protocols for treating disorders in human patients. For example, sequences derived from regions adjacent to the intron/exon boundaries of the human gene can be used to design primers for use in amplification assays to detect mutations within the exons or introns that can be used in diagnostics.

Furthermore, a growth factor gene homolog may be isolated from nucleic acid of the organism of interest by performing amplification procedures using two degenerate oligonucleotide primer pools designed on the basis of amino acid sequences within the growth factor gene product disclosed herein. For the purpose of this invention, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, Taq polymerase, pfu polymerase and/or RNA polymerases such as reverse transcriptase. A preferred amplification method is PCR. General procedures for PCR are taught in U.S. Pat. Nos. 4,683195 (Mullis et al.) and 4,683,202 (Mullis et al.). However, optimal PCR conditions used for each application reaction are generally empirically determined or estimated with computer software commonly employed by artisans in the field. A number of parameters influence the success of a reaction. Among them are annealing temperature and time, extension time, Mg²⁺ ATP concentration, pH, and the relative concentration of primers, templates, and deoxyribonucleotides.

The template for the reaction may be cDNA obtained by reverse transcription of mRNA prepared from cell lines or tissue known or suspected to express a growth factor gene allele. The PCR product may be subcloned and sequenced to ensure that the amplified sequences represent the sequences of a growth factor gene. The PCR fragment may then be used to isolate a full length cDNA clone by a variety of methods. For example, the amplified fragment may be labeled and used to screen a cDNA library, such as a bacteriophage cDNA library. Alternatively, the labeled fragment may be used to isolate genomic clones via the screening of a genomic library.

Amplification procedures can also be utilized to isolate full-length cDNA sequences. A representative amplification technique applicable for gene cloning is 5′-RACE-PCR. In this technique, the poly-A mRNA that contains the coding sequence of particular interest is first identified by hybridization to a sequence disclosed herein and then reverse transcribed with a 3′-primer comprising the sequence disclosed herein. The newly synthesized cDNA strand is then tagged with an anchor primer of a known sequence, which preferably contains a convenient cloning restriction site attached at the 5′end. The tagged cDNA is then amplified with the 3′-primer (or a nested primer sharing sequence homology to the internal sequences of the coding region) and the 5′-anchor primer. The amplification may be conducted under conditions of various levels of stringency to optimize the amplification specificity. 5′-RACE-PCR can be readily performed using commercial kits (available from, e.g., BRL Life Technologies Inc, Clotech) according to the manufacturer's instructions.

The exemplary growth factor gene sequences can also be employed to isolate mutant growth factor gene alleles. Such mutant alleles may be isolated from individuals either known or proposed to have a genotype that contributes to the symptoms of disorders arising from the aberrant expression or activity of the growth factor protein. See U.S. Pat. No. 5,837,832. By comparing the DNA sequence of the mutant growth factor allele to that of the normal growth factor allele, the mutation(s) responsible for the loss or alteration of function of the mutant growth factor gene product can be ascertained. Mutant alleles and mutant allele products may then be utilized in the therapeutic and diagnostic systems described herein. Additionally, such growth factor gene sequences can be used to detect growth factor gene regulatory (e.g., promoter or promoter/enhancer) defects that can affect the expression of the growth factor.

The polynucleotides embodied in this invention can be conjugated with a detectable label. Such polynucleotides are useful, for example, as probes for detection of related nucleotide sequences. Detectable labels suitable for use in the present invention include any composition detectable by spectroscopic, photochemical, biochemical, immunochemical, electrical, optical or chemical means. A wide variety of appropriate detectable labels are known in the art, which include luminescent labels, radioactive isotope labels, enzymes or other ligands. In preferred embodiments, one will likely desire to employ a fluorescent label or an enzyme tag, such as digoxigenin, β-galactosidase, urease, alkaline phosphatase or peroxidase, avidin/biotin complex. The labels may be incorporated by any of a number of means well known to those of skill in the art. In one aspect, the label is simultaneously incorporated during the amplification step in the preparation of the invention polynucleotides. Thus, for example, polymerase chain reaction (PCR) with labeled primers or labeled nucleotides can provide a labeled amplification product. In a separate aspect, transcription reaction, as described above, using a labeled nucleotide (e.g. fluorescein-labeled UTP and/or CTP, digoxigenin-UTP) or a labeled primer, incorporates a detectable label into the transcribed nucleic acids.

Alternatively, a label may be added directly to the original polynucleotide sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by phosphorylation of the polynucleotides with a kinase and subsequent attachment (ligation) of a nucleic acid linker to a label (e.g., a fluorophore).

The polynucleotides of this invention can be obtained by chemical synthesis, recombinant cloning, e.g. PCR, or any combination thereof. Methods of chemical polynucleotide synthesis are well known in the art and need not be described in detail herein. For example, devices such as the ABI Model 3948 nucleic acid synthesizer available from Perkin Elmer/Applied Biosystems, Inc., Foster City, Calif., USA are commercially available for oligonucleotide synthesis. One of skill in the art can use the sequence data provided herein to obtain a desired polynucleotide by employing a DNA synthesizer, PCR machine, or ordering from a commercial service.

### USES OF THE POLYNUCLEOTIDES OF THE PRESENT INVENTION

The polynucleotides of this invention have several uses. Growth factor polynucleotides are useful, for example, in expression systems for the production of growth factor or growth factor fragments. They are also useful as hybridization probes to clone the full-length growth factor cDNA as described above, or to assay for the presence of growth factor sequences in a sample using methods well known to those in the art. The hybridization assays employing such probes have numerous applications including but not limited to growth factor gene expression analysis, fingerprinting, sequence mapping, growth factor chromosomal localization and polymorphism detection. See U.S. Pat. No. 5,800,992. Furthermore, the polynucleotides are useful as primers to effect amplification of desired polynucleotides. The polynucleotides of this invention are also useful in antibody production, disease diagnosis, prognosis, and treatment.

Methods for conducting the aforementioned genetic analysis using the polynucleotides of the present invention are well known to artisans in the field (see Sambrook, supra), and hence are not detailed herein. Briefly, representative techniques include, microarray assays (e.g. U.S. Pat. Nos. 5,445,934 and 5,800,992 Fodor et al.), amplification procedures (e.g. PCR, RT-PCR), FISH, SAGE (Velculescu, et al. (1995) Science 270:484-487 and U.S. Pat. No. 5,695,937 Kinzler et al.), in-situ hybridization, nucleotide sequencing, restriction fragment length polymorphism (RFLP), single-strand conformation polymorphism assay, and allele-specific oligonucleotide hybridization.

### VECTORS, HOST CELLS, AND TRANSGENIC ORGANISMS OF THE PRESENT INVENTION

The polynucleotides of the present invention can be inserted into a suitable gene delivery vehicle, and the vehicle in turn can be introduced into a suitable host cell for replication and amplification. Accordingly, this invention further provides a variety of gene delivery vehicles comprising the polynucleotide of the present invention. Gene delivery vehicles include both viral and non-viral vectors. Non-limiting examples of gene delivery vehicles are liposomes, plasmid, bacteriophage, cosmid, fungal vectors, viruses, such as adenovirus, baculovirus, and retrovirus, and any other recombination vehicles capable of carrying an inserted polynucleotide into a host cell.

Vectors are generally categorized into cloning and expression vectors. Cloning vectors are useful for obtaining replicate copies of the polynucleotides they contain, or as a means of storing the polynucleotides in a depository for future recovery. Expression vectors (and host cells containing these expression vectors) can be used to obtain polypeptides produced from the polynucleotides they contain. Suitable cloning and expression vectors include any known in the art, e.g., those for use in bacterial, mammalian, yeast and insect expression systems. The polypeptides produced in the various expression systems are also within the scope of the invention.

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will grow under selective conditions. Typical selection genes either: (a) confer resistance to antibiotics or other toxins, e.g., ampicillin, neomycin, methotrexate; (b) complement auxotrophic deficiencies; or (c) supply critical nutrients not available from complex media. The choice of the proper marker gene will depend on the host cell, and appropriate genes for different hosts are known in the art. Vectors also typically contain a replication system recognized by the host.

Suitable cloning vectors can be constructed according to standard techniques, or selected from a large number of cloning vectors available in the art. While the cloning vector selected may vary according to the host cell intended to be used, useful cloning vectors will generally have the ability to self-replicate, may possess a single target for a particular restriction endonuclease, or may carry marker genes. Suitable examples include plasmids and bacterial viruses, e.g., pBR322, pMB9, Co1E1, pCR1, RP4, pUC18, mp18, mp19, phage DNAs, and shuttle vectors such as pSA3 and pAT28. These and other cloning vectors are available from commercial vendors such as Stratagene, Clontech, BioRad, and Invitrogen.

Expression vectors containing the growth factor polynucleotides are useful to obtain host vector systems to produce growth factor polypeptides. It is implied that these expression vectors must be replicable in the host organisms either as episomes or as an integral part of the chromosomal DNA. Typically, the growth factor polynucleotide of interest is operably linked to a regulatory element that directs the expression of the growth factor polypeptide. As used herein, regulatory elements include but are not limited to inducible and non-inducible promoters, enhancers, operators and other elements known to those skilled in the art that drive and regulate expression. Such regulatory elements include, but are not limited to, the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

A variety of host-expression vector systems may be utilized to express a growth factor polynucleotide of the invention. Where the growth factor peptide or polypeptide is soluble, the peptide or polypeptide can be recovered from the culture. Such systems are desirable where it is important not only to retain the structural and functional characteristics of the growth factor, but to assess biological activity, e.g., in drug screening assays. However, the expression systems also encompass engineered host cells that express the growth factor or functional equivalents attached to or embedded in cell membranes. Purification or enrichment of the growth factor from such expression systems can be accomplished using appropriate detergents and lipid micelles and methods well known to those skilled in the art.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing growth factor polynucleotides; yeast (e.g., Saccharomyces, Pichia) transformed with recombinant yeast expression vectors containing the growth factor polynucleotides; insect cell systems infected with recombinant virus expression vectors (e.g., baculovirus) containing the growth factor sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing growth factor polynucleotides; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the growth factor gene product being expressed. For example, when a large quantity of such a protein is to be produced, for the generation of pharmaceutical compositions of growth factor protein or for raising antibodies to the growth factor protein, for example, vectors which direct the expression of high levels of fusion protein products that are readily purified may be desirable. Such vectors include, but are not limited, to the E. coli expression vector pUR278 (Ruther et al. (1983) EMBO J. 2:1791-1794), in which the growth factor coding sequence may be ligated individually into the vector in frame with the lacZ coding region so that a fusion protein is produced; pIN vectors (Inouye and Inouye, (1985) Nucleic Acids Res. 13:3101- 3110; Van Heeke and Schuster (1989) J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. The growth factor gene coding sequence may be cloned individually into non-essential regions (for example the polyhedrin gene) of the virus and placed under control of an AcNPV promoter (for example the polyhedrin promoter). Successful insertion of growth factor gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g, see, Schroeder et al., U.S. Pat. No. 4,215,051).

In mammalian host cells, a number of viral-based expression systems may be utilized. In cases where an adenovirus is used as an expression vector, the growth factor nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the growth factor gene product in infected hosts. (E.g., See Logan and Shenk (1984) Proc. Natl. Acad. Sci. USA 81:3655-3659). Specific initiation signals may also be required for efficient translation of inserted growth factor nucleotide sequences. These signals include the ATG initiation codon and adjacent sequences. In cases where an entire growth factor gene or cDNA, including its own initiation codon and adjacent sequences, is inserted into the appropriate expression vector, no additional translational control signals may be needed. However, in cases where only a portion of the growth factor coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bitter et al. (1987) Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. In addition, substitution of rare codons for common codons encoding the same amino acid may be utilized to increase polypeptide production in a particular species. For instance, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3 and WI38.

For long-term, high-yield production of recombinant proteins, stable expression is preferred. For example, cell lines which stably express the growth factor sequences described above may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the growth factor gene product. Such engineered cell lines may be particularly useful in screening and evaluation of compounds that affect the endogenous activity of the growth factor gene product.

A number of selection systems may be used, including but not limited to the herpes simplex virus thymidine kinase (Wigler et al. (1977) Cell 11:223-232), hypoxanthine-guanine phosphoribosyltransferase, and adenine phosphoribosyltransferase (Lowy, et al. (1980) Cell 22:817-823) genes can be employed in tk⁻, hgprt⁻ or aprt⁻ cells, respectively. Also, antimetabolite resistance can be used as the basis of selection for the following genes: dhfr, which confers resistance to methotrexate (Wigler et al. (1980) Proc. Natl. Acad. Sci. USA 77:3567-3570; Hare et al. (1981) Proc. Natl. Acad. Sci. USA 78:1527-1531); gpt, which confers resistance to mycophenolic acid (Mulligan and Berg (1981) Proc. Natl. Acad. Sci. USA 78:2072-2076); neo, which confers resistance to the aminoglycoside G-418 (Colbere-Garapin et al. (1981) J. Mol. Biol. 150:1-14); and hygro, which confers resistance to hygromycin (Santerre et al. (1984) Gene 30:147-156).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al. (1991) Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺. nitriloacetic acid-agarose columns and histidine-tagged proteins are selectively eluted with imidazole-containing buffers.

The growth factor gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate growth factor transgenic animals.

Any technique known in the art may be used to introduce the growth factor transgene into animals to produce the founder lines of transgenic animals. Such techniques include, but are not limited to pronuclear microinjection (Hoppe, P. C. and Wagner, T. E., U.S. Pat. No. 4,873,191); retrovirus mediated gene transfer into germ lines (van der Putten et al. (1985) Proc. Natl. Acad. Sci., USA 82:6148-6152); gene targeting in embryonic stem cells (Thompson et al. (1989) Cell 56:313-321); electroporation of embryos (Lo (1983) Mol Cell. Biol. 3:1803-1814); and sperm-mediated gene transfer (Lavitrano et al. (1989) Cell 57:717-723); etc. For a review of such techniques, see Gordon (1989) Transgenic Animals, Intl. Rev. Cytol. 115:171 -229, which is incorporated by reference herein in its entirety.

The present invention provides for transgenic animals that carry the growth factor transgene in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al. (1992) Proc. Natl. Acad. Sci. USA 89: 6232-6236). The regulatory sequences required for such a cell-type specific activation will depend upon the particular cell type of interest, and will be apparent to those of skill in the art. When it is desired that the growth factor gene transgene be integrated into the chromosomal site of the endogenous growth factor gene, gene targeting is preferred. Briefly, when such a technique is to be utilized, vectors containing some nucleotide sequences homologous to the endogenous growth factor gene are designed for the purpose of integrating, via homologous recombination with chromosomal sequences, into and disrupting the function of the nucleotide sequence of the endogenous growth factor gene. The transgene may also be selectively introduced into a particular cell type, thus inactivating the endogenous growth factor gene in only that cell type, by following, for example, the teaching of Gu et al. (Gu, et al. (1994) Science 265: 103-106). The regulatory sequences required for such a cell-type specific inactivation will depend upon the particular cell type of interest, and will be apparent to those of skill in the art.

Once transgenic organisms have been generated, the expression of the recombinant growth factor gene may be assayed utilizing standard techniques. Initial screening may be accomplished by microarray analysis or PCR techniques to analyze tissues of the transgenic organism to assay whether integration of the transgene has taken place. The level of mRNA expression of the transgene in the tissues of the transgenic organism may also be assessed using techniques which include but are not limited to Northern blot analysis of tissue samples obtained from the organism, in situ hybridization analysis, and RT-PCR. Samples of growth factor gene-expressing tissue, may also be evaluated immunocytochemically using antibodies specific for the growth factor transgene product.

### POLYPEPTIDES AND ANTIBODIES OF THE PRESENT INVENTION

This invention encompasses proteins or polypeptides expressed from the polynucleotides of this invention, which are intended to include wild-type, chemically synthesized and recombinantly produced polypeptides and proteins from prokaryotic and eukaryotic host cells, as well as muteins, analogs and fragments thereof. In some embodiments, the term also includes various types of antibodies that specifically bind to the growth factor polypeptides.

Also encompassed by this embodiment are proteins functionally equivalent to the growth factor encoded by the polynucleotides described in the aforementioned section. A “functional equivalent” varies from the wild-type sequence by any combination of addition, deletion, or substitution while preserving at least one functional property of the invention growth factor relevant to the context in which it is being tested. Relevant growth factor functional properties include but are not limited to the ability of the equivalent polypeptide to bind natural growth factor receptor, the ability to affect the action of downstream molecules such as heterotrimeric G proteins, the ability to elicit growth factor cellular responses including, e.g. ion flux, mobilization of Ca²⁺ from intracellular stores, tyrosine or serine phosphorylation, or change in cellular phenotype when the growth factor equivalent is present in an appropriate cell type. Such functionally equivalent growth factor proteins may contain amino acid substitutions introduced on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Whereas random mutations can be introduced to growth factor polynucleotides (using methods well established in the art), and the resulting mutant growth factors tested for growth factor activity, site-directed mutation is preferred to generate mutant growth factors with an altered growth factor functional property or expression profile. These alterations in growth factor function may evince an increase in binding affinity for natural growth factor receptor, and/or greater signaling capacity; or a decrease in receptor binding affinity and/or signal transduction capacity; or differential expression of growth factor in various body tissues or intracellular compartments.

In designing mutant growth factors, it is preferable to discern the boundaries of various domains that are conserved amongst homologs from selected species. For example, regions of identity may be determined by alignment of growth factor with growth factor homologs from other species or through structural analysis of high-resolution structure images of growth factor homologs from other species. Mutant growth factors can be engineered so that regions of identity are maintained, whereas the variable residues are altered, e.g., by addition, deletion or insertion of one or more conservative amino acid residue(s). Where alteration of function is desired, deletion or non-conservative alterations of the conserved regions can be engineered. For example, deletion or non-conservative alterations (substitutions or insertions) of the receptor binding domain can be introduced so as to alter the binding affinity of the resulting growth factor. Other mutations to the growth factor coding sequence can be made to generate growth factors that are better suited for expression, scale up, etc. in the host cells chosen.

Growth factors have a characteristic carboxy-terminal growth factor homology region containing several cysteine residues which form disulfide bonds to maintain unique tertiary structure. Polypeptides corresponding to this growth factor domain, truncated or deleted growth factors (e.g., growth factor in which one or more cysteines are deleted) as well as fusion proteins in which the full length growth factor, a growth factor peptide or truncated growth factor is fused to an unrelated protein, or one or more domains of a growth factor of a different class, are also within the scope of the invention. Useful fusion partners include sequences that enhance immunological reactivity or the coupling of the polypeptide to an immunoassay support of a vaccine carrier. As such, the resulting fusion proteins include but are not limited to (a) immunoglobulin fusions which stabilize the growth factor protein or peptide and prolong half-life in vivo; (b) fusions to unrelated proteins or epitopes (e.g. GST, flu-tagg, myc-tag, FLAG-tag) to facilitate protein purification; (c) fusions to a signal sequence that directs the fusion protein to the cell membrane, (d) fusions to the growth factor homology domain of a different class of growth factor, so as to alter the binding specificity of the fusion growth factor, or to effect shunting of downstream signaling to a different pathway, which may be more amenable to a high throughput analysis.

The polypeptides of the invention can also be conjugated to a chemically functional moiety. Typically, the moiety is a label capable of producing a detectable signal. These conjugated polypeptides are useful, for example, in detection systems for diagnosis and screening assays described herein. A wide variety of labels are known in the art. Non-limiting examples of the types of labels which can be used in the present invention include radioisotopes, enzymes, colloidal metals, and luminescent compounds.

The polypeptides of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides.

The polypeptides of this invention can be prepared by a number of processes well known to those of skill in the art. Representative techniques are purification, chemical synthesis and recombinant methods. Cellular growth factor can be purified from tissues or cells expressing the growth factor by methods such as immunoprecipitation with antibody, and standard techniques such as gel filtration, ion-exchange, reversed-phase, and affinity chromatography using a fusion protein as shown herein. For such methodology, see for example Deutscher et al., GUIDE TO PROTEIN PURIFICATION: METHODS IN ENZYMOLOGY, v.182 (1999).

Alternatively, the polypeptides also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). In addition, the invention polypeptides can be generated recombinantly by expressing polynucleotides using the vector systems and host cells as described in the section above.

This invention further provides antibodies that specifically bind to one or more epitopes of growth factor, or epitopes of conserved variants of growth factor, or peptide fragments of the growth factor. Such antibodies include but are not limited to polyclonal antibodies, monoclonal antibodies (mAbs), Fab, Fab′, F(ab′)₂ fragments, humanized or chimeric antibodies, single chain antibodies, anti-idiotypic (anti-Id) antibodies, and epitope-binding fragments of any of the above.

The production of these antibodies and epitope-binding fragments are well established in the art. For instance, Fab fragments may be generated by digesting a whole antibody with papain and contacting the digest with a reducing agent to reductively cleave disulfide bonds. Fab′ fragments may be obtained by digesting the antibody with pepsin and reductive cleavage of the fragment so produce with a reducing agent. In the absence of reductive cleavage, enzymatic digestion of the monoclonal with pepsin produces F(ab′)₂ fragments. Alternatively, Fab fragments can be recombinantly produced by a Fab expression library (see e.g. Huse et al. (1989) Science, 246:1275-1281).

For production of polyclonal antibodies, an appropriate host animal is immunized with substantially purified growth factor polypeptide, whether the full-length growth factor, mutant growth factor, functional equivalents, fusion growth factor, or a fragment of any of the above. Suitable host animals may include but are not limited to mouse, rabbits, mice, and rats. The growth factor polypeptide is introduced commonly by injection into the host footpads, via intramuscular, intraperitoneal, or intradermal routes. Peptide fragments suitable for raising antibodies may be prepared by chemical synthesis, and are commonly coupled to a carrier molecule (e.g., keyhole limpet hemocyanin), or admixed with adjuvants to enhance the immunogenicity of the antigen. Depending on the host species, suitable adjuvants can be Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as BCG (bacille Calmette-Guerin) and Corynebacterium parvum.

Sera harvested from the immunized animals provide a source of polyclonal antibodies. Detailed procedures for purifying specific antibody activity from a source material are known within the art. Undesired activity cross-reacting with other antigens, if present, can be removed, for example, by running the preparation over adsorbents made of those antigens attached to a solid phase and eluting or releasing the desired antibodies off the antigens. If desired, the specific antibody activity can be further purified by such techniques as protein A chromatography, ammonium sulfate precipitation, ion exchange chromatography, high-performance liquid chromatography and immunoaffinity chromatography on a column of the immunizing polypeptide coupled to a solid support.

The generation of monoclonal antibodies, which are homogeneous populations of antibodies to a particular antigen, can be carried out by any technique which provides for the production of antibody molecules by continuous cell lines in culture. These include, but are not limited to, the hybridoma technique of Kohler and Milstein (1975) Nature 256:495-497 and U.S. Pat. No. 4,376,110, the human B-cell hybridoma technique, and the EBV-hybridoma technique (Cole et al., MONOCLONAL ANTIBODIES AND CANCER THERAPY, at 77-96 (1985)).

Monoclonal antibodies to the growth factor can, in turn, be utilized to generate anti-idiotype antibodies (Greenspan & Bona (1993) FASEB J 7(5):437-444; and Nisonoff (1991) J. Immunol. 147(8):2429-2438), which recognize unique epitopes present on the monoclonal antibody. Of particular interest is the type of anti-idiotype antibodies that “mimic” the growth factor epitope which is recognized by the parent monoclonal antibody. For instance, the parent monoclonal antibody capable of binding to the growth factor receptor binding domain, and competitively inhibiting the binding of natural growth factor receptor to the growth factor can be used to generate anti-idiotypes that “mimic” the receptor binding domain and, therefore, bind and neutralize natural growth factor receptor. Such neutralizing anti-idiotypes or Fab fragments of such anti-idiotypes are particularly useful in therapeutic regimens to neutralize the physiological activity of the natural growth factor receptor.

Also encompassed in this embodiment are “chimeric antibodies” in which various portions are derived from different animal species. A “humanized antibody” is a type of chimeric antibody in which all regions except the antigen binding portions (also referred to as “CDRs”) are derived from a non-human species. Such antibody can be produced by fusing the constant regions of the heavy and light chains of a human immunoglobulin with the variable regions of a murine antibody that confirm the antigen-binding specificity. See e.g. Morrison et al. (1984) Proc. Natl. Acad. Sci., 81:6851-6855; Neuberger et al. (1984) Nature, 312:604-608; Takeda et al. (1985) Nature, 314:452-454. A variation of this approach is to replace residues outside the antigen-binding domains of a non-human antibody with the corresponding human sequences (see WO 94/11509). Another approach for production of human monoclonal antibodies is the use of xenogenic mice as described in U.S. Pat. No. 5,814,318 (Lonberg et al.) and U.S. Pat. No. 5,939,598 (Kucherlapati et al.). These genetically engineered mice are capable of expressing certain unrearranged human heavy and light chain immunoglobulin genes, with their endogenous immunoglobulin genes being inactivated.

In addition, techniques have been developed for the generation of single chain antibodies (U.S. Pat. No. 4,946,778 (Ladner et al.); Bird (1988) Science 242:423-426; Huston et al. (1988) Proc. Natl. Acad. Sci. USA 85:5879-5883; and Ward et al. (1989) Nature 341:544-546). Single chain antibodies are formed by linking the heavy and light chain fragments of the Fv region via an amino acid bridge, resulting in a single chain polypeptide.

The specificity of an antibody refers to the ability of the antibody to distinguish polypeptides comprising the immunizing epitope from other polypeptides. An ordinary skill in the art can readily determine without undue experimentation whether an antibody shares the same specificity as a antibody of this invention by determining whether the antibody being tested prevents an antibody of this invention from binding the polypeptide(s) with which the antibody is normally reactive. If the antibody being tested competes with the antibody of the invention as shown by a decrease in binding by the antibody of this invention, then it is likely that the two antibodies bind to the same or a closely related epitope. Alternatively, one can pre-incubate the antibody of this invention with the polypeptide(s) with which it is normally reactive, and determine if the antibody being tested is inhibited in its ability to bind the antigen. If the antibody being tested is inhibited, then, in all likelihood, it has the same, or a closely related, epitopic specificity as the antibody of this invention.

The antibodies of the invention can be bound to many different carriers. Accordingly, this invention also provides compositions containing antibodies and a carrier, which can be active or inert. Examples of well-known carriers include polypropylene, polystyrene, polyethylene, dextran, nylon, amylases, glass, natural and modified celluloses, polyacrylamides, agaroses and magnetite. See U.S. Pat. No. 5,445,934. The nature of the carrier can be either soluble or insoluble for purposes of the invention. Those skilled in the art will know of other suitable carriers for binding antibodies, or will be able to ascertain such, using routine experimentation.

The antibodies of this invention can also be conjugated to a detectable agent or a hapten. The complex is useful to detect the polypeptide(s) containing the recognized epitopes to which the antibody specifically binds in a sample, using standard immunochemical techniques such as immunohistochemistry as described by Harlow and Lane (1988). supra. A wide diversity of labels and methods of labeling are known to those of ordinary skill in the art. Representative labels that can be employed in the present invention include radioisotopes, enzymes, colloidal metals, and luminescent compounds. Those of ordinary skill in the art will know of other suitable labels for binding to the antibody, or will be able to ascertain such, using routine experimentation.

The antibodies of the invention may be used, for example, in the detection of the growth factor in a biological sample and may, therefore, be utilized as part of a diagnostic or prognostic technique whereby patients may be tested for abnormal amounts of growth factor. Such antibodies may also be utilized in conjunction with, for example, compound screening schemes, as described below, for the evaluation of the effect of test compounds on expression and/or activity of the growth factor gene product. In addition, such antibodies can be used as therapeutics for restoring normal or inhibiting aberrant growth factor response in a cell.

### SCREENING ASSAYS FOR GROWTH FACTOR RECEPTORS AND MODULATORS

A wealth of studies has shown that growth factors play a central role in a variety of physiological processes. Defects in various components of growth factor signal transduction pathways have been found to account for a vast number of diseases, including numerous forms of cancer, vascular diseases, metabolic diseases, immunological, and neuronal diseases. Indeed, receptors and modulators of growth factor activity have long been acknowledged as potential diagnostic and/or therapeutic agents.

Accordingly, the present invention provides a method for identifying receptors or modulators of a growth factor encoded by the polynucleotide disclosed herein. The method involves the steps of (a) contacting a candidate growth factor receptor or modulator with said growth factor; and (b) assaying for an alteration in G protein response and/or growth factor expression.

For the purposes of this invention, a “modulator” is intended to include, but not be limited to biological or chemical molecules that interact with (e.g., bind to) growth factor or its receptor, molecules that bind to transmembrane proteins that interact with growth factor or its receptor, molecules that interfere with the interaction of growth factor with it receptor, or other transmembrane or intracellular proteins (e.g. heterotrimeric G proteins) involved in growth factor-mediated signal transduction, and molecules which modulate the activity of growth factor gene or expression profile. Of particular interest are modulators capable of binding to the growth factor and either mimic the activity triggered in the natural receptor (i.e., agonists) or inhibit the activity triggered in the natural receptor (i.e., antagonists), or “neutralize” the activity of the natural receptor. Also of particular significance are modulators which interact with growth factor gene regulatory sequences (e.g., promoter sequences) so as to regulate growth factor gene expression (see, e.g. Platt, K. A. (1994) J. Biol. Chem. 269:28558-28562).

Candidate modulators for the present invention include a biological or chemical compound such as a simple or complex organic or inorganic molecule. Such compounds may include, but are not limited to, peptides such as, for example, soluble peptides, including but not limited to members of random peptide libraries; (see, e.g., Lam, K. S. et al. (1991) Nature 354:82-84; Houghten, R. et al. (1991) Nature 354:84-86), and combinatorial chemistry-derived molecular library made of D- and/or L- configuration amino acids, phosphopeptides (including, but not limited to, members of random or partially degenerate, directed phosphopeptide libraries; see e.g., Songyang, Z. et al. (1993) Cell 72:767-778); molecules from natural product libraries, antibodies (including, but not limited to, polyclonal, monoclonal, humanized, anti-idiotypic, chimeric or single chain antibodies, and FAb, F(ab′)₂ and FAb expression library fragments, and epitope-binding fragments thereof). In addition, a vast array of small organic or inorganic compounds from natural sources such as plant or animal extracts, and the like, can be employed in the screening assay. It should be understood, although not always explicitly stated that the modulator is used alone or in combination with another modulator, having the same or different biological activity as the modulators identified by the inventive screen.

While crude receptor signaling studies can be performed outside of cells using, for example, reconstituted vesicles (see e.g., J. R. Hepler (1996) J. Biol. Chem. 271: 496-504), it is preferable to employ cell-based functional assays. A preferred cell-based method for identifying growth factor receptors or modulators generally includes the following steps: (a) providing a host cell expressing, or preferably over-expressing a growth factor of interest; (b) exposing the growth factor to a candidate growth factor modulator or cell-based receptor, and (c) detecting an alteration in growth factor response or expression within the contact cell.

Suitable conditions to allow binding of a modulator to a growth factor are physiological conditions wherein the pH is maintained between 6 and 8, and the temperature is between about 20-40 degrees C. As used herein, the binding of a modulator to growth factor is understood to denote an interaction of the molecule with any portions of the growth factor polypeptide, which may result in a conformational change in topology of the polypeptide. The binding of the modulator to a growth factor polypeptide may either trigger (in the case of agonist) or block (in the case of antagonist) a detectable growth factor response. Such growth factor responses include but are not limited to (a) activation of phospholipase C proteins; (b) increase in phosphatidylinositol (PI) hydrolysis; (c) increase in intracellular calcium; (d) inhibition of the adenyl cyclase activity; and (e) activation of adenyl cyclase activity resulting in transient or more permanent accumulation of intracellular cAMP.

Methods of measuring intracellular inositol phosphates are well known in the art. Briefly, cell membrane phospholipids can be labeled by incubating host cells with [³H] myo-inositol for 20-24 hours. Cells are then stimulated with appropriate modulators. Cell extracts can be collected and inositol phosphates separated by ion-exchange chromatography (e.g., by using AG1-X8 in either the chloride- or formate-form; when only IP₃ levels are to be determined, the chloride-form is preferably used, whereas the formate form can be used to resolve the major inositol phosphates (IP₃, IP₂ and IP₁).

Measuring intracellular calcium fluctuation can be rapidly accomplished with the use of calcium-sensitive fluorescent probes, including but not limited to Fura-2, Fluo-3 and Calcium Green-1. Changes of calcium level are reflected by a change in fluorescence of these probes, which can be measured by a high throughput assay that is adaptable to robotic processing. For example, host cells loaded with fluorescent probes can be monitored by FLIPR (Molecular Devices Corp.), an instrument capable of performing stimulation in all 96 wells of samples contained in a microplate simultaneously, and providing real-time measurement and functional data once every second. Typically, the assay is completed in less than fifteen minutes. Since more than a hundred 96-microplates can be read in a day, nearly 10,000 different compounds can be tested for growth factor agonist or antagonist. A variety of cell types, both adherent and non-adherent, can be used in FLIPR.

Another exemplary high throughput assay for measuring intracellular calcium or cAMP content involves induction of a reporter gene operatively linked to a calcium-responsive or cAMP-responsive element (e.g. promoter sequence). In this method, a calcium flux or cAMP accumulation resulting from the activation of growth factor turns on the promoter which subsequently drives the expression of a reporter gene encoding a protein with an enzymatic activity that can be easily detected, preferably by a colorimetric or fluorescent assay. Commonly used reporter proteins include: β-galactosidase, β-lactamase, chloramphenicol acetyltransferase (CAT), luciferase, green fluorescent protein (GFP) and its derivatives, among others. Reporter proteins can also be linked to other proteins whose expression is dependent upon the stimulation of growth factors. An illustrative example would be a fusion protein comprising luciferase sequence in frame with the open-reading frame of nuclear factor of activated T cells (NFAT). Since the transcription of NFAT requires the co-activation of calcium and protein kinases C signaling pathways acting downstream of growth factors, an effective coupling of heterotrimeric G protein to the receptors can then be measured by assaying NFAT-mediated luciferase activity (Boss et al. (1996) J. Biol. Chem., 271: 10429- 10432). In practice of this method, a preferred host cell is one of lymphoid or neuronal origin, such as Jurkat cells and pheochromocytoma PC12 cells. However, the choice of host cells is not limited to these two types, as NFAT and NFAT isoforms are present in a variety of cells including endothelial and myeloid cells.

Other screening techniques include the use of cells which express the growth factor receptor (for example, transfected CHO cells) in a system which measures extracellular pH changes caused by receptor activation by the growth factor. In addition, a cytosensor microphysiometer can be used to detect and monitor the response of cells to chemical substances as described in McConnell et al. (1992) Science 257: 1906-1912. For example, potential agonists or antagonists may be interact with the growth factor which contacts a cell expressing the growth factor receptor and a second messenger response, e.g., signal transduction or pH changes, may be measured to determine whether the potential agonist or antagonist is effective.

Another such screening technique involves introducing RNA encoding growth factor receptor into Xenopus oocytes to transiently express the receptor. The receptor expressing oocytes may then be contacted, in the case of antagonist screening, with the growth factor of the invention and a compound to be screened, followed by detection of inhibition of a calcium signal.

Screening for antagonists can also be carried out by a contacting a candidate antagonist in the presence of labeled growth factor to cells which express the receptor on the surface. The amount of labeled growth factor bound to the receptors is inversely proportional to the ability of the candidate antagonist to inhibit growth factor binding. Thus, a reduced amount of labeled growth factor bound to the receptor indicates that the antagonist is effective in inhibiting the binding of the natural growth factor to the receptor.

A variety of in vitro assays are also available in the art to identify modulators of the present invention. In general, the in vitro assays are performed by contacting the growth factor polypeptide with a candidate modulator under conditions that will allow a complex to form between the growth factor and the modulator. The formation of the complex can be detected directly or indirectly according standard procedures in the art. In the direct detection method, the modulators are supplied with a detectable label and unreacted modulators may be removed from the complex; the amount of remaining label thereby indicating the amount of complex formed. For such method, it is preferable to select labels that remain attached to the modulators even during stringent washing conditions. It is more important, however, that the label does not interfere with the binding reaction. In the alternative, an indirect detection procedure requires the modulators to contain a label introduced either chemically or enzymatically, that can be detected by affinity cytochemistry. A desirable label generally does not interfere with target binding or the stability of the resulting complex. However, the label is typically designed to be accessible to an antibody for an effective binding and hence generating a detectable signal. A wide variety of labels are known in the art. Non-limiting examples of the types of labels which can be used in the present invention include radioisotopes, enzymes, colloidal metals, fluorescent compounds, bioluminescent compounds, and chemiluminescent compounds.

The amount of modulator-growth factor complex formed during the binding reaction can be quantified by standard quantitative assays. As illustrated above, the formation of modulator-growth factor complex can be measured directly by the amount of label remaining at the site of binding. In an alternative, the modulator is tested for its ability to compete with a labeled growth factor for binding sites on the specific receptor. In this competitive assay, the amount of label captured is inversely proportional to the ability of the modulator to compete for receptor binding.

Modulators such as transmembrane proteins or intracellular proteins capable of interacting with the growth factor or its receptor can be identified by a vast diversity of in vitro or in vivo techniques that are well established in the art. Among the conventional methods are co-immunoprecipitation, crosslinking and co-purification through gradients or chromatographic columns of cell lysates or proteins obtained from cell lysates. For these assays, the growth factor component used can be a full length growth factor, a peptide corresponding to receptor binding domain of the growth factor. Once isolated, such an intracellular protein can be identified and can, in turn, be used, in conjunction with standard techniques, to identify proteins with which it interacts. For example, at least a portion of the amino acid sequence of an intracellular protein which interacts with the growth factor receptor can be ascertained using techniques well known to those of skill in the art, such as via the Edman degradation technique or other peptide mapping techniques.

In an alternative, growth factor interacting proteins can be isolated by yeast two-hybrid system as illustrated by Chien et al. (1991) Proc. Natl. Acad. Sci. USA, 88:9578-9582. This hybrid system is also commercially available from Clontech (Palo Alto, Calif.).

In addition to compounds affecting growth factor activities or cellular responses, modulators capable of regulating the growth factor gene expression are also encompassed within this embodiment. Alteration of gene expression can be determined by examining the growth factor protein product or growth factor mRNA level.

Determining the protein level involves (a) providing a biological sample containing polypeptides; and (b) measuring the amount of any immunospecific binding that occurs between an antibody reactive to the protein products of interest and a component in the sample, in which the amount of immunospecific binding indicates the level of the protein products.

Biological samples used for this invention encompass body fluid, solid tissue samples, tissue cultures or cells derived therefrom and the progeny thereof, and sections of smears prepared from any of these sources.

A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunoflourescent assays, and SDS-PAGE. In addition, cell sorting analysis can be employed to detect cell surface antigens. Such analysis involves labeling target cells with antibodies coupled to a detectable agent, and then separating the labeled cells from the unlabeled ones in a cell sorter. A sophisticated cell separation method is fluorescence-activated cell sorting (FACS). Cells traveling in single file in a fine stream are passed through a laser beam, and the fluorescence of each cell bound by the fluorescently labeled antibodies is then measured.

Antibodies that specifically recognize and bind to the protein products of interest are required for conducting the aforementioned protein analyses. Anti-growth factor antibodies can be generated by the methods disclosed under the “Antibody section” or other methods well known in the art. See Harlow and Lane (1988) supra. and Sambrook et al. (2001) supra.

To determine a change in the growth factor mRNA level in a cell, hybridization assays employing the invention polynucleotides is generally performed. Nucleic acid contained in the aforementioned biological samples is first extracted according to standard methods in the art. For instance, mRNA can be isolated using various lytic enzymes or chemical solutions according to the procedures set forth in Sambrook et al. (2001), supra or extracted by nucleic-acid-binding resins following the accompanying instructions provided by manufactures. The mRNA contained in the extracted nucleic acid sample is then detected by hybridization and/or amplification procedures according to methods widely known in the art or based on the methods disclosed herein. See U.S. Pat. No. 5,800,992.

### DIAGNOSTICS AND THERAPEUTICS OF THE PRESENT INVENTION

The polynucleotides, polypeptides, antibodies, receptors, and modulators of this invention provide specific reagents that can be used in standard diagnostic, or prognostic evaluation of cardiovascular, neuronal, metabolic, and immunological disorders. These reagents may be used, for example, for: (a) the detection of the presence of growth factor gene mutations, or the detection of differential expression of growth factor mRNA or protein product relative to the non-disorder state; (b) the detection of perturbations or abnormalities in the signal transduction pathway signaled by the growth factor. Techniques for genetic analyses and protein analyses are described in the sections above. Provided with these critical reagents for detecting the growth factor polynucleotides and polypeptides, one skilled in the art can readily perform various diagnostic procedures without undue experimentation. Where desired, a normal or standard growth factor expression profile or activity range can be established for a comparative diagnosis.

The polynucleotides, polypeptides, antibodies, receptors, and modulators of this invention may be employed as therapeutics for treatment of growth factor associated diseases. Based on the chemical and structural homology among the invention growth factors and previously characterized growth factors including various members of the Family 3946 growth factors, the growth factor of this invention is expected to play a role in the regulation of a variety of biological pathways involving e.g. ion channel (e.g. calcium channel), protein kinases, proteases and many other second messengers in a cell. Dysfunction of these cellular components have been found to account for a vast number of diseases, including numerous forms of cancer, diabetes and other pancreatic diseases, osteoporosis, immunological, vascular diseases, neuronal diseases, hypercalcemia, and hyperparathyroid.

Accordingly, in one aspect, growth factor anti-sense polynucleotides can be administered to a subject to treat a disease correlated with an abnormally high level of growth factor expression, such as in many forms of cancer including breast cancer and lung cancer. Conversely, sense-strand growth factor can be delivered to and expressed in a subject suffering from a disease correlated with an aberrantly low level of growth factor expression, such as diseases of early development.

In another aspect, growth factor polypeptides, antibodies, antigen-binding fragments, and modulators that function as agonists can be administered to stimulate the endogenous growth factor activity where such a stimulation is appropriate, for example, to promote cell proliferation during wound healing possibly following surgery.

In another aspect, growth factor polypeptides, antibodies, antigen-binding fragments, and modulators that function as antagonists can be administered to a subject exhibiting an abnormal high level of endogenous growth factor activity.

The present invention encompasses pharmaceutical compositions containing growth factor polynucleotides, polypeptides, vectors, receptors, modulators, antibodies, fragments thereof, and/or cell lines which produce the antibodies or fragments. Such pharmaceutical compositions are useful for eliciting an immune response and treating growth factor associated diseases, either alone or in conjunction with other forms of therapy, such as chemotherapy or radiotherapy.

The preparation of pharmaceutical compositions of this invention is conducted in accordance with generally accepted procedures for the preparation of pharmaceutical preparations. See e.g. REMINGTON'S PHARMACEUTICAL SCIENCES 18TH EDITION (1990), E. W. Martin, ed. Depending on the intended use and mode of administration, it may be desirable to process the active ingredient further in the preparation of pharmaceutical compositions. Appropriate processing may include sterilizing, mixing with appropriate non-toxic and non-interfering components, dividing into dose units, and enclosing in a delivery device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

Pharmaceutical compositions of the present invention are administered by a mode appropriate for the form of composition. Typical routes include subcutaneous, intramuscular, intraperitoneal, intradermal, oral, intranasal, and intrapulmonary (i.e., by aerosol). Pharmaceutical compositions of this invention for human use are typically administered by a parenteral route, most typically intracutaneous, subcutaneous, or intramuscular.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, pharmaceutical compositions are preferably supplied in unit dosage form suitable for administration of a precise amount. Also contemplated by this invention are slow release or sustained release forms, whereby a relatively consistent level of the active compound are provided over an extended period.

### KITS COMPRISING THE POLYNUCLEOTIDES OF THE PRESENT INVENTION

The present invention also encompasses kits containing the polynucleotides, polypeptides, antibodies, antigen-binding fragments and vectors of this invention in suitable packaging. Kits embodied by this invention include those that allow someone to detect the presence or quantify the amount of growth factor polynucleotide or polypeptide that is suspected to be present in a sample. The sample is optionally pre-treated for enrichment of the target being tested for. The user then applies a reagent contained in the kit in order to detect the changed level or alteration in the diagnostic component.

Each kit necessarily comprises the reagent which renders the procedure specific: a reagent antibody or polynucleotide probe or primer, used for detecting target protein and polynucleotide, respectively. Each reagent can be supplied in a solid form or dissolved/suspended in a liquid buffer suitable for inventory storage, and later for exchange or addition into the reaction medium when the test is performed. Suitable packaging is provided. The kit can optionally provide additional components that are useful in the procedure. These optional components include, but are not limited to, buffers, capture reagents, developing reagents, labels, reacting surfaces, means for detection, control samples, instructions, and interpretive information. The kits can be employed to test a variety of biological samples, including body fluid, solid tissue samples, tissue cultures or cells derived therefrom and the progeny thereof, and sections or smears prepared from any of these sources. Diagnostic procedures using the antibodies of this invention can be performed by diagnostic laboratories, experimental laboratories, practitioners, or private individuals.

### COMPUTER-READABLE MEDIA OF THE PRESENT INVENTION

The present invention provides a computer readable medium having recorded the polynucleotide and/or polypeptides of the present invention. As used herein, a “computer readable medium” refers to any medium which can be read and accessed directly by a computer. Such media include, but are not limited to magnetic storage media, such as floppy discs, hard disc storage medium, and magnetic tape; optical storage media such as CD-ROM; electrical storage media such as RAM and ROM; and hybrids of these categories, such as magnetic/optical storage media. A skilled artisan can readily appreciate how any of the presently known computer readable mediums can be used to create a manufacture comprising compute readable medium having recorded thereon the polynucleotides and/or polypeptides of the present invention. Likewise, it will be clear to those of skill how additional computer readable media that may be developed also can be used to create analogous manufactures having recorded thereon the invention polynucleotides and/or polypeptides encoded thereby.

The term “recorded” refers to a process for storing information on computer readable medium. A skilled artisan can readily adopt any of the presently known methods for recording information on computer readable medium to generate manufactures comprising polynucleotides or polypeptides of the present invention. A variety of data storage structures are available to a skilled artisan for creating a computer readable medium having recorded thereon the nucleotide or amino acid sequence information of this invention. The choice of the data storage structure will generally be based on the means chosen to access the stored information. In addition, a variety of data processor programs and formats can be used to store the sequence information of the present invention on a computer readable medium. For instance, the sequence information can be stored in a file using ASCII or other binary formats. Where desired, files stored under ASCII format can be subcategorized into comma delimited file, tab delimited file, space delimited file, and the like. Non-limiting exemplary binary formats other than ASCII include Microsoft Word, Word Perfect, Excel, and Adobe Acrobat. In addition, the sequence formation can be stored in certain database format including but not limited to DB2, Sybase, Oracle, Informix, SQL or the like. A skilled artisan can readily adapt any number of data-processing software formats in order to obtain computer readable medium having recorded thereon the sequence information of the present invention.

We claim:

1. An isolated polynucleotide comprising a nucleic acid sequence shown in FIG. 1B.

2. An isolated polynucleotide comprising a nucleic acid sequence selected from the group consisting of:
(a) a nucleic acid sequence of at least 90 nucleotides that is essentially identical to a linear nucleotide sequence of comparable length depicted in FIG. 1B;
(b) a nucleic acid sequence of at least 90 nucleotides encoding a polypeptide that is essentially identical to a linear peptide sequence of at least 30 amino acids depicted in FIG. 1A; and
(c) a complement of (a) or (b).

3. The isolated polynucleotide of claim 2 wherein said nucleic acid is (a).

4. The isolated polynucleotide of claim 2 wherein said nucleic acid is (b).

5. The isolated polynucleotide of claim 2 wherein said nucleic acid is (c).

6. The isolated polynucleotide of claim 2 wherein said nucleic acid encodes a polypeptide comprising an amino acid sequence that is essentially identical to a linear sequence of comparable length shown in FIG. 1A.

7. The isolated polynucleotide of claim 2 wherein said nucleic acid sequence encodes a polypeptide comprising the amino acid sequence shown in FIG. 1A.

8. The isolated polynucleotide of claim 2 wherein said nucleic acid encodes a polypeptide comprising an amino acid sequence essentially identical to the entire amino acid sequence shown in FIG. 1A.

9. The isolated polynucleotide of claim 2 wherein said nucleic acid is identical to a linear nucleotide sequence of comparable length contained in the sequence shown in FIG. 1B.

10. The isolated polynucleotide of claim 2 which is DNA.

11. The isolated polynucleotide of claim 2 which is RNA.

12. The isolated polynucleotide of claim 10, wherein the DNA is a full-length cDNA molecule.

13. The isolated polynucleotide of claim 2 further comprising a heterologous polynucleotide.

14. The isolated polynucleotide of claim 13, wherein the heterologous polynucleotide encodes a heterologous polypeptide.

15. A pharmaceutical composition comprising the polynucleotide of claim 1.

16. The isolated polynucleotide of claim 1, wherein said polynucleotide is conjugated with a detectable label selected from the group consisting of enzymes, radioactive moieties and luminescent moieties.

17. A gene delivery vehicle, comprising an isolated polynucleotide of claim 1.

18. The gene delivery vehicle of claim 17, wherein the vehicle is selected from the group consisting of viral vector, a liposome and a plasmid.

19. A genetically engineered host cell comprising an isolated polynucleotide of claim 1.

20. A recombinant method of producing a polypeptide that comprises culturing the genetically engineered host cell of claim 19 under conditions suitable for protein expression, and isolating the expressed polypeptide.

21. An isolated polypeptide encoded by the polynucleotide of claim 1.

22. A pharmaceutical composition comprising the polypeptide of claim 21.

23. An antibody that specifically binds to the isolated polypeptide of claim 21.

24. The antibody of claim 23, wherein the antibody is a monoclonal antibody.

25. A hybridoma cell line that produces the monoclonal antibody of claim 24.

26. The antibody of claim 24, wherein the monoclonal antibody is a humanized antibody.

27. A method for identifying a modulator of the growth factor encoded by the polynucleotide of claim 1, comprising:
(a) contacting a candidate modulator with said growth factor; and
(b) assaying for an alteration of growth factor activity and/or growth factor expression.

28. The method of claim 27, where the growth factor activity is characterized by a stimulation of phospholipase C activity.

29. The method of claim 27, where the growth factor activity is characterized by a stimulation or an inhibition of adenyl cyclase activity.

30. The method of claim 27, wherein the candidate modulator is selected from the group consisting of an antisense oligonucleotide, a ribozyme, a ribozyme derivative, an antibody, a liposome, a small molecule and an inorganic compound.

31. A modulator identified by the method of claim 27.

32. A method for identifying a receptor of the growth factor encoded by the polynucleotide of claim 1, comprising:
(a) contacting a candidate receptor with said growth factor; and
(c) assaying for an alteration of growth factor activity and/or growth factor expression.

33. The method of claim 32, wherein the contacting step occurs in a cell comprising said receptor.

34. The method of claim 32, where the growth factor activity is characterized by a stimulation of phospholipase C activity.

35. The method of claim 27, where the growth factor activity is characterized by a stimulation or an inhibition of adenyl cyclase activity.

36. A receptor identified by the method of claim 32.

37. A method of diagnosing a pathogenic condition or susceptibility to a pathogenic condition that is associated with a genetic alteration in the growth factor encoded by the polynucleotide of claim 1, comprising:
(a) providing a biological sample of a subject containing nucleic acid molecules and/or polypeptides;
(b) determining a genetic alteration associated with the growth factor; and
(c) correlating the alteration with a pathogenic condition or susceptibility to a pathogenic condition.

38. The method of claim 37, wherein the genetic alteration is selected from the group consisting of sequence deletion, substitution, translocation, and differential gene expression.

39. A computer readable medium having recorded thereon the nucleic acid sequence of claim 1.

40. A computer readable medium having recorded thereon the polypeptide sequence of claim 21.

41. The computer readable medium of claim 39 or 40, wherein said medium is selected from the group consisting of:
(a) magnetic storage medium;
(b) optical storage medium;
(c) electrical storage medium; and
(d) hybrid storage medium of (a), (b), (c) or (d).

42. A computer readable medium of claim 41, wherein the magnetic storage medium is selected from the group consisting of floppy discs, hard disc, and magnetic tape.

43. A computer readable medium of claim 41, wherein the optical storage medium is CD-ROM.

44. A computer readable medium of claim 41, wherein the electrical storage media is random access memory (RAM) or read only memory (ROM).

45. A computer readable medium of claim 41, wherein the hybrid storage medium is magnetic/optical storage medium.

46. A transgenic animal comprising the gene delivery vehicle of claim 17.

47. A kit comprising the isolated polynucleotide of claim 1 in suitable packaging.

48. A kit comprising the isolated polypeptide of claim 21 in suitable packaging.
